(19) United States
(12) Patent Application Publication
(10) Pub. No.: US 2003/0049640 A1
(43) Pub. Date: Mar. 13, 2003

(54) Proteins, genes and their use for diagnosis and treatment of vascular response

(76) Inventors: Eugene Herman, Takoma Park, MD (US); Gordon Duane Holt, Gaithersburg, MD (US); Frank Sistare, Ellicott City, MD (US); Jun Zhang, Rockville, MD (US)

Correspondence Address: PENNIE AND EDMONDS, 1155 AVENUE OF THE AMERICAS, NEW YORK, NY, 100362711

(21) Appl. No.: 10/033,741

(22) Filed: Dec. 27, 2001

Related U.S. Application Data
(60) Provisional application No. 60/260,387, filed on Dec. 29, 2000.

(51) Int. Cl. C12Q 1/68; G01N 33/53; G01N 33/542; G01N 33/557

(52) U.S. Cl. 435/6; 435/7.9; 436/517

(57) ABSTRACT

The present invention provides methods and compositions for screening, diagnosis and prognosis of vascular response, for monitoring the effectiveness of vascular response treatment, identifying patients most likely to respond to a particular therapeutic treatment and for drug development. In particular, the screening of drug candidates for their ability to induce a vascular response. Vascular Response-Associated Features (VRFs), detectable by two-dimensional electrophoresis of blood, serum or plasma are described. The invention further provides Vascular Response-Associated Protein Isoforms (VRPIs) detectable in blood, serum or plasma, preparations comprising isolated VRPIs, antibodies immunospecific for VRPIs, and kits comprising the aforesaid.

### 1. INTRODUCTION

The present invention relates to the identification of proteins and protein isoforms that are associated with vascular response to endogenous and exogenous effector agents, including its onset and development, and of genes encoding the same, and to their use for clinical screening, diagnosis, prognosis, therapy and prophylaxis, as well as for drug screening and drug development.

### 2. BACKGROUND OF THE INVENTION

Blood vessels, including the veins, arteries and capillaries, play an essential role in the distribution of molecules throughout the body. The blood vessels also circulate a variety of cells (e.g., immune cells, erythrocytes, platelets, bone marrow and stem cells). The cells lining the blood vessels respond to the circulating molecules and cells, which are defined herein as “endogenous and exogenous effector agents” for example by rapidly modulating metabolic pathways and by expressing new patterns of protein export and cell surface expression. Any disruptions in vascular responsiveness to environmental changes can lead to serious, often life-threatening, consequences. A wide variety of effector agents are provided herein as examples of, but are not limited to, inducers of vascular responses:

Endogenous Effector Agents

Acute disruptive agents

infarct

ischemia

thrombus

Chronic disruptive agents

diabetes

hypercholesterolemia

hypertension

physical inactivity

poor nutrition

overweightness/obesity

Genetic disease

Carotid Artery Disease

Cushing's disease

Lymphedema

Thoracic Outlet Syndrome

Vasculititdes (e.g., Wegeners disease)

Exogenous Effector Agents

Xenobiotics

Viruses

Chemical agents and medications

nicotine & smoking

chemotherapeutics (e.g., a variety of approved and experimental treatments for indications such as cancer)

coagulants and anticoagulants

vasoconstrictors and vasodialators

drugs of abuse (e.g., cocaine, amphetamines)

neovascularization effector molecules (e.g., metaloprotease inhibitors, proteoglycans)

Trauma

accidental

surgery (e.g., bypass, angioplasty)

Blood vessels are architecturally complex and composed of many unique cell types. Vascular-disrupting effector agents may exclusively affect just one of these cell types, or, more commonly, may interfere with several types simultaneously. Thus, affected areas may range from highly focal to diffuse lesions, and may spread or refocus over time. A wide variety of vascular changes are defined herein as examples of, but not limited to, vascular responses to effector agents: Aneurysm; Artherosclerosis; Congestion; Edema; Hemorrhage; Shock; Stenosis;StrokeVaricose veins; andVasculitis (angiitis).

The following list outlines currently validated measures of vascular responses to one or more of the agents described above:

Nonintrusive assays

blood pressure monitoring

exercise/stress test

soft tissue imaging including doppler flow ultrasonography, magnetic resonance imaging, computed tomography

angiography

Intrusive assays

needle biopsy

surgery

All of these current measures of vascular response suffer from one or more significant limitations. For example, the non-intrusive assays show poor correlation with vascular histopathology and generally provide no prospective measure of how the vascular response will change over time. The intrusive vascular response assays also suffer from the limitation that they present significant risk to the test subject. Therefore, they generally are not employed unless the subject's life is already under serious threat. In addition, the intrusive assays require time-consuming and costly interpretation by expert pathologists, and may also provide ambiguous results if the tissue changes are not homogeneous across the blood vessels relative to the sample examined.

The current measures of vascular response are also severely limited in their usefulness in facilitating the development of new treatments for human disease.

Some anecdotal studies have shown that the levels of certain blood or blood vessel cell proteins change in response to exposure to effector. However, we are unaware of any of these studies systematically encompassing effector treatments over several time points or progression of response to identify statistically significant changes in protein levels.

Due to the costly and time consuming nature of existing, often ambiguous, tests it would be highly desirable to measure a substance or substances in samples of blood or blood vessel cells that would lead to a positive diagnosis of vascular response or that would help to exclude vascular response from a differential diagnosis.

The development of new pharmaceutical compositions and/or treatment regimens directed towards the treatment or prophylaxis of diseases, infectious or otherwise, relies heavily on the ability to screen candidate compounds for possible toxic or pathological responses, e.g. vascular response. In drug development, a putative drug is tested in a battery of assays and in laboratory animals to ascertain its safety (i.e. lack of toxicity) and effectiveness. The costs associated with the development of new pharmaceutical reagents are ever increasing, particularly when new compositions enter clinical trials. It is not unheard of for promising pharmaceutical candidates to pass the appropriate laboratory tests and enter the expensive stage of animal and human clinical trials, only to present toxic or pathologic effects in the in vivo setting for the targeted patient, normally humans. The elimination of previously-promising drug candidates at such a late stage in product development is a major factor in the high costs of new effective drugs which ultimately do pass the final clinical trials.

Therefore, a need exists to identify vascular response-associated proteins as sensitive and specific biomarkers for the diagnosis, to assess severity and predict the outcome of vascular response in response subjects and, in particular, to allow the screening of drug candidates for their ability to induce a vascular response. Additionally, there is a clear need for new therapeutic agents for vascular response that work quickly, potently, specifically, and with fewer side effects.

### 3. SUMMARY OF THE INVENTION

The present invention provides methods and compositions for clinical screening, diagnosis, prognosis, therapy and prophylaxis of vascular response, in particular, the screening of drug candidates for their ability to induce a vascular response, for monitoring the effectiveness of vascular response treatment, for selecting participants in clinical trials, for identifying patients most likely to respond to a particular therapeutic treatment and for screening and development of drugs for treatment of vascular response.

A first aspect of the invention provides methods for diagnosis of vascular response that comprise analyzing a sample of body fluid, e.g. blood, by two-dimensional electrophoresis to detect the presence or level of at least one Vascular Response-Associated Feature (VRF), e.g., one or more of the VRFs disclosed herein or any combination thereof. These methods are also suitable for clinical screening, prognosis, monitoring the results of therapy, identifying patients most likely to respond to a particular therapeutic treatment, for drug screening and development, and identification of new targets for drug treatment.

A second aspect of the invention provides methods for diagnosis ofvascular response that comprise detecting in a sample of body fluid, e.g.blood, the presence or level of at least one Vascular Response-Associated Protein Isoform (VRPI), e.g., one or more of the VRPIs disclosed herein or any combination thereof. These methods are also suitable for clinical screening, prognosis, monitoring the results of therapy, identifying patients most likely to respond to a particular therapeutic treatment, drug screening and development, and identification of new targets for drug treatment.

A third aspect of the invention provides antibodies, e.g. monoclonal and polyclonal antibodies capable of immunospecific binding to a VRPI, e.g. a VRPI disclosed herein.

A fourth aspect of the invention provides a preparation comprising an isolated VRPI, i.e., a VRPI free from proteins or protein isoforms having a significantly different isoelectric point or a significantly different apparent molecular weight from the VRPI.

A fifth aspect of the invention provides kits that may be used in the above recited methods and that may comprise single or multiple preparations, or antibodies, together with other reagents, labels, substrates, if needed, and directions for use. The kits may be used for diagnosis of disease, or may be assays for the identification of new diagnostic and/or therapeutic agents.

A sixth aspect of the invention provides methods of treating vascular response, comprising administering to a subject a therapeutically effective amount of an agent that modulates (e.g., upregulates or downregulates) the expression or activity (e.g. enzymatic or binding activity), or both, of aVRF, a VRPI in subjects having vascular response, in order to prevent or delay the onset or development of vascular response, to prevent or delay the progression of vascular response, or to ameliorate the symptoms of vascular response.

A seventh aspect of the invention provides methods of screening for agents that modulate (e.g., upregulate or downregulate) a characteristic of, e.g., the expression or the enzymatic or binding activity, of a VRF, a VRPI, a VRPI analog, or a VRPI-related polypeptide. This aspect of the invention being particularly useful in determining the ability of drug candidiates to induce a vascular response.

### 4. BRIEF DESCRIPTION OF THE FIGURES

FIG. 1 is a flow chart depicting the characterization of a VRF and relationship of a VRF and VRPI. A VRF may be further characterized as or by a VRPI having a particular peptide sequence associated with its pI and MW. As depicted herein, a VRF may comprise one or more VRPIs, which have indistinguishable pI and MWs using the Preferred Technology, but which comprise distinct peptide sequences. The peptide sequence(s) of the VRPI can be utilized to search database(s) for previously-identified proteins comprising such peptide sequence(s). In some instances, it can be ascertained whether a commercially-available antibody exists which may recognize the previously identified protein and/or a variant thereof. It should be noted that the VRPI may correspond to the previously-identified protein, be a variant of the previously identified protein, or be a previously unknown protein.

FIG. 2 is an image obtained from 2-dimensional electrophoresis of plasma, which has been annotated to identify twelve landmark features, designated F1 to F11 and F13, and which are illustrative of an embodiment of an aspect of the present invention.

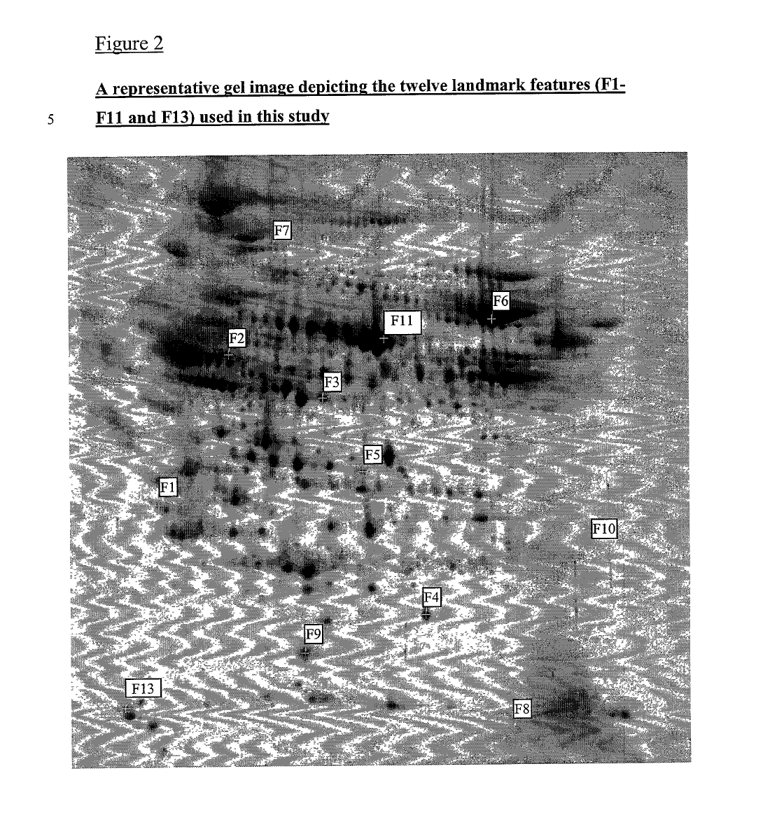

### 5. DETAILED DESCRIPTION OF THE INVENTION

The present invention described in detail below provides methods, compositions and kits useful, e.g., for screening, diagnosis and treatment of Vascular Response in a mammalian subject, and for drug screening and drug development. When the invention is used to determine the ability of drug candidiates to induce a vascular response, the body fluid which is analysed for the presence or level of at least one vascular response feature is preferably from a non-human mammal. The non-human mammal is preferably one in which the induction of a vascular response by endogenous and/or exogenous effector agents is predictive of the induction of such a response in humans. The rat is a particularly suitable mammal for use in this aspect of the invention.

The invention also encompasses the administration of therapeutic compositions to a mammalian subject to treat or prevent Vascular Response. The mammalian subject may be a non-human mammal, but is preferably human, more preferably a human adult, i.e. a human subject at least 21 (more particularly at least 35, at least 50, at least 60, at least 70, or at least 80) years old. For clarity of disclosure, and not by way of limitation, the invention will be described with respect to the analysis of plasma samples. However, as one skilled in the art will appreciate, based on the present description the assays and techniques described below can be applied to other types of samples, including a body fluid (e.g. blood, serum, plasma, or saliva), a tissue sample from a subject at risk of having or developing Vascular Response (e.g. a biopsy of vascular tissue) or homogenate thereof from a subject at risk of having or developing Vascular Response. The methods and compositions of the present invention are useful, such as for example, screening, diagnosis and treatment of a living subject, but may also be used for postmortem diagnosis in a subject, for example, to identify family members of the subject who are at risk of developing the same disease.

The following definitions are provided to assist in the review of the instant disclosure.

### 5.1. Definitions

“Vascular Response” refers to and includes the alteration in blood and blood vessels and/or any condition, that comes about from the interaction of the blood or blood vessels with endogenous and/or exogenous effector agents, including xenobiotics, viruses or other biological agents, particularly those agents listed as effector agents, which can generate a Vascular Response or which otherwise reduce or alter the function or physiological reponse of the blood vessels. Vascular Response encompasses and includes those activities, alterations and physiological occurances in the blood vessels or otherwise associated with the blood or the blood vessel function, which take place during any alteration of the blood vessels including but not limited to aneurysm, artherosclerosis, congestion, edema, hemorrhage, shock, stenosis, stroke, varicose veins and vasculitis (angiitis). Endogenous effector agents include but are not limited to infarct, ischemia, thrombus, diabetes, hypercholesterolemia, hypertension, physical inactivity, poor nutrition, overweightness/obesity and genetic disease (e.g. carotid artery disease, Cushing's disease, lymphedema, thoracic outlet syndrome, vasculititdes (e.g., Wegeners disease). Exogenous effector agents include but are not limited to xenobiotics (e.g. SKF-95654 as particularly utilized herein), viruses, nicotine & smoking, chemotherapeutics (e.g., a variety of approved and experimental treatments for indications such as cancer), coagulants and anticoagulants, vasoconstrictors and vasodialators, drugs of abuse (e.g., cocaine, amphetamines), neovascularization effector molecules (e.g., metaloprotease inhibitors, proteoglycans), accidental trauma and surgery (e.g., bypass, angioplasty).

“Feature” refers to a spot detected in a 2D gel, and the term “Vascular Response-Associated Feature” (VRF) refers to a feature that is differentially present in a sample from a subject having a Vascular Response compared with a sample from a subject free from a Vascular Response. A feature or spot detected in a 2D gel is characterized by its isoelectric point (pI) and molecular weight (MW) as determined by 2D gel electrophoresis, particularly utilizing the Preferred Technology described herein. As used herein, a feature is “differentially present” in a first sample with respect to a second sample when a method for detecting the said feature (e.g., 2D electrophoresis) gives a different signal when applied to the first and second samples. A VRF, (or a protein isoform, i.e. VRPI, as defined infra) is “increased” in the first sample with respect to the second if the method of detection indicates that the VRF, or VRPI is more abundant in the first sample than in the second sample, or if the VRF, or VRPI is detectable in the first sample and substantially undetectable in the second sample. Conversely, a VRF, or VRPI is “decreased” in the first sample with respect to the second if the method of detection indicates that the VRF, or VRPI is less abundant in the first sample than in the second sample or if the VRF, or VRPI is undetectable in the first sample and detectable in the second sample.

Preferably, the relative abundance of a feature in two samples is determined in reference to its normalized signal, in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature whose abundance is substantially invariant, within the limits of variability of the Preferred Technology, in the population of subjects being examined, e.g. the ERFs disclosed below, or (c) more preferably to the total signal detected as the sum of each of all proteins in the sample.

Secondly, the normalized signal for the feature in one sample or sample set is compared with the normalized signal for the same feature in another sample or sample set in order to identify features that are “differentially present” in the first sample (or sample set) with respect to the second.

“Fold change” includes “fold increase” and “fold decrease” and refers to the relative increase or decrease in abundance of a VRF or the relative increase or decrease in expression or activity of a polypeptide (e.g. a VRPI, as defined infra.) in a first sample or sample set compared to a second sample (or sample set). A VRF or polypeptide fold change may be measured by any technique known to those of skill in the art, albeit the observed increase or decrease will vary depending upon the technique used. Preferably, fold change is determined herein as described in the Examples infra.

“Vascular Response-Associated Protein Isoform” (VRPI) refers to a protein that is differentially present in a sample from a subject having a Vascular Response compared with a sample from a subject free from any Vascular Response or that is differentially present in a sample from a subject having one or more particular Vascular Response compared with a sample from a subject free from such one or more particular Vascular Response or having a distinct Vascular Response. As used herein, a VRPI is “differentially present” in a first sample with respect to a second sample when a method for detecting the said feature, (e.g., 2D electrophoresis or immunoassay) gives a different signal when applied to the first and second samples (refer to VRF definition).

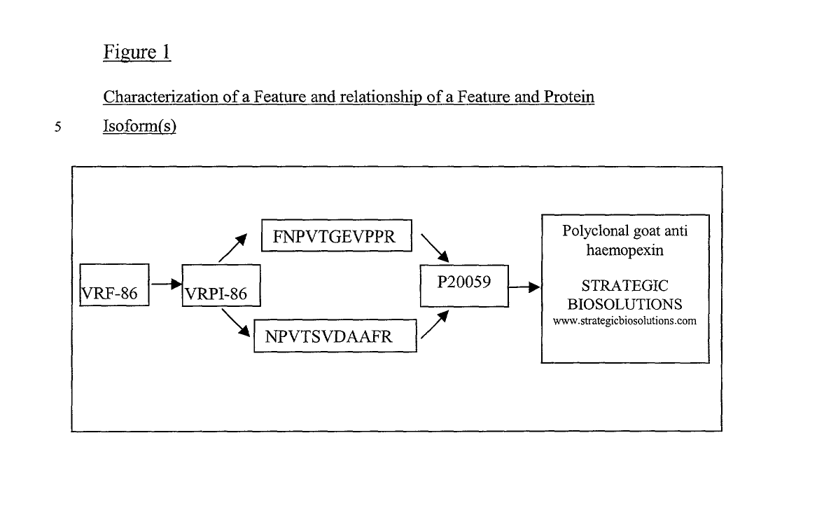

A VRPI is characterized by one or more peptide sequences of which it is comprised, and further by a pI and MW, preferably determined by 2D gel electrophoresis, particularly utilizing the Preferred Technology as described herein. Typically, VRPIs are identified or characterized by the amino acid sequencing of VRFs (FIG. 1).

FIG. 1 is a flow chart depicting the characterization of a VRF and relationship of a VRF and VRPI. A VRF may be further characterized as or by a VRPI having a particular peptide sequence associated with its pI and MW. As depicted herein, a VRF may comprise one or more VRPIs, which have indistinguishable pI and MWs using the Preferred Technology, but which comprise distinct peptide sequences. The peptide sequence(s) of the VRPI can be utilized to search database(s) for previously-identified proteins comprising such peptide sequence(s). In some instances, it can he ascertained whether a commercially-available antibody exists which may recognize the previously identified protein and/or a variant thereof. It should be noted that the VRPI may correspond to the previously-identified protein, be a variant of the previously identified protein, or be a previously unknown protein.

“Variant” as used herein refers to a polypeptide which is a member of a family of polypeptides that are encoded by a single gene or from a gene sequence within a family of related genes and which differ in their pI or MW, or both. Such variants can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation).

“Modulate” in reference to expression or activity of a VRPI or a VRPI-related polypeptide refers to any change, e.g., upregulation or downregulation, of the expression or activity of the VRPI or a VRPI-related polypeptide. Those skilled in the art, based on the present disclosure, will understand that such modulation can be determined by assays known to those of skill in the art.

“VRPI analog” refers to a polypeptide that possesses similar or identical function(s) as a VRPI but need not necessarily comprise an amino acid sequence that is similar or identical to the amino acid sequence of the VRPI, or possess a structure that is similar or identical to that of the VRPI. As used herein, an amino acid sequence of a polypeptide is “similar” to that of a VRPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the VRPI; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the VRPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the VRPI. As used herein, a polypeptide with “similar structure” to that of a VRPI refers to a polypeptide that has a similar secondary, tertiary or quarternary structure as that of the VRPI. The structure of a polypeptide can determined by methods known to those skilled in the art, including but not limited to, X-ray crystallography, nuclear magnetic resonance, and crystallographic electron microscopy.

“VRPI fusion protein” refers to a polypeptide that comprises (i) an amino acid sequence of a VRPI, a VRPI fragment, a VRPI-related polypeptide or a fragment of a VRPI-related polypeptide and (ii) an amino acid sequence of a heterologous polypeptide (i.e., a non-VRPI, non-VRPI fragment or non-VRPI-related polypeptide). “VRPI homolog” refers to a polypeptide that comprises an amino acid sequence similar to that of a VRPI but does not necessarily possess a similar or identical function as the VRPI.

“VRPI ortholog” refers to a non-rat polypeptide that (i) comprises an amino acid sequence similar to that of a VRPI and (ii) possesses a similar or identical function to that of the VRPI. It will be appreciated that the specific VRPIs identified in the description were derived from the rat. The skilled person will recognise that in various aspects of the invention it will be necessary to substitute the rat VRPI with the VRPI ortholog from another mammal e.g. a human. VRPI orthologs can be identified using techniques well known to those skilled in the art for example using homology searching e.g. as described below in relation to the determination of per cent identitiy of two amino acid sequences. The similarity between the VRPIs identified and their human orthologs is on average 85% (S.E.M.=2.6) allowing for conservative substitutions (see section 5.7). It will be appreciated that in various aspects of the claimed invention, e.g. methods of treatment, it will be necessary to substitute a VRPI with a VRPI ortholog depending on the identity of the mammal to be treated.

“VRPI-related polypeptide” refers to a VRPI homolog, a VRPI analog, a variant of VRPI, a VRPI ortholog, or any combination thereof.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.)

“Derivative” refers to a polypeptide that comprises an amino acid sequence of a second polypeptide that has been altered by the introduction of amino acid residue substitutions, deletions or additions. The derivative polypeptide possesses a similar or identical function as the second polypeptide.

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of a VRPI may or may not possess a functional activity of the second polypeptide.

The “percent identity” of two amino acid sequences or of two nucleic acid sequences can be or is generally determined by aligning the sequences for optimal comparison purposes (e.g., gaps can be introduced in either sequences for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=#of identical positions/total#of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search.

“Diagnosis” refers to diagnosis, prognosis, monitoring, characterizing, selecting patients, including participants in clinical trials, and identifying patients at risk for or having a particular disorder or those most likely to respond to a particular therapeutic treatment, or for assessing or monitoring a patient's response to a particular therapeutic treatment.

“Treatment” refers to therapy, prevention and prophylaxis and particularly refers to the administration of medicine or the performance of medical procedures with respect to a patient, for either prophylaxis (prevention) or to cure the infirmity or malady in the instance where the patient is afflicted.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Blood” as used herein includes serum and plasma. “Serum” refers to the supernatant fluid produced by clotting and centrifugal sedimentation of a blood sample; “plasma” refers to the supernatant fluid produced by inhibition of clotting (for example, by citrate or EDTA) and centrifugal sedimentation of a blood sample.

As used herein, the term “vascular tissue” refers to the cell layers that line veins, arteries and capillaries.

### 5.2 Vascular Response-Associated Features (YRFs)

In one aspect of the invention, two-dimensional electrophoresis is used to analyze plasma from a subject, preferably a living subject, in order to detect or quantify the expression of one or more Vascular Response-Associated Features (VRFs) for screening, treatment or diagnosis of Vascular Response, to determine the prognosis of a subject having vascular response, to monitor progression of vascular response, to monitor the effectiveness of vascular response therapy, for identifying patients most likely to respond to a particular therapeutic treatment, or for drug development, and ,in particular, to determine the potential for drug candidaites to induce a vascular response.

By way of example and not of limitation, using the Preferred Technology, a number of samples from subjects having a Vascular Response and samples from subjects free from a Vascular Response are separated by two-dimensional electrophoresis, and the fluorescent digital images of the resulting gels are matched to a chosen representative primary master gel image. This process allows any gel feature, characterised by its pI and MW, to be identified and examined on any gel of the study. In particular, the amount of protein present in a given feature can be measured in each gel; this feature abundance can be averaged amongst gels from similar samples (e.g. gels from samples from subjects having a Vascular Response). Finally, statistical analyses can be conducted on the thus created sample sets, in order to compare 2 or more sample sets to each other.

As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in International Application No. 97GB3307 (published as WO 98/23950) and in U.S. Pat. No 6,064,754, both filed Dec. 1, 1997, each of which is incorporated herein by reference in its entirety with particular reference to the experimental protocol. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A preferred scanner for detecting fluorescently labeled proteins is described in WO 96/36882 and in the Ph.D. thesis of David A. Basiji, entitled “Development of a High-throughout Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

As used herein, the term “feature” refers to a spot detected in a 2D gel, and the term “Vascular Response-Associated Feature” (VRF) refers to a feature that is differentially present in a sample (e.g. a sample of blood) from a subject having vascular response compared with a sample (e.g. a sample of blood) from a subject free from vascular response. As used herein, a feature (or a protein isoform of VRPI, as defined infra) is “differentially present” in a first sample with respect to a second sample when a method for detecting the feature, isoform or VRPI (e.g., 2D electrophoresis or an immunoassay) gives a different signal when applied to the first and second samples. A feature, isoform or VRPI is “increased” in the first sample with respect to the second if the method of detection indicates that the feature, isoform or VRPI is more abundant in the first sample than in the second sample, or if the feature, isoform or VRPI is detectable in the first sample and undetectable in the second sample. Conversely, a feature, isoform or VRPI is “decreased” in the first sample with respect to the second if the method of detection indicates that the feature, isoform or VRPI is less abundant in the first sample than in the second sample or if the feature, isoform or VRPI is undetectable in the first sample and detectable in the second sample.

Preferably, the relative abundance of a feature in two samples is determined in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature whose abundance is invariant, within the limits of variability of the Preferred Technology, in the population of subjects being examined, e.g. the ERFs disclosed below, or (c) more preferably to the total signal detected from all proteins in the sample.

Secondly, the normalized signal for the feature in one sample or sample set is compared with the normalized signal for the same feature in another sample or sample set in order to identify features that are “differentially present” in the first sample (or sample set) with respect to the second.

In accordance with an aspect of the present invention, the VRFs disclosed herein have been identified by comparing blood samples from subjects having vascular response against blood samples from subjects free from vascular response. Subjects free from vascular response include subjects with no known disease or condition (normal subjects) and subjects with diseases other than vascular response.

Vascular response was induced by xenobiotic treatment as described in the Examples infra, and samples were analyzed 1 hour, 2 hours, 4 hours and 24 hours following treatment.

Two groups of VRFs have been identified through the methods and apparatus of the Preferred Technology.The first group consists of VRFs that are decreased in the blood of subjects having vascular response as compared with the blood of subjects free from vascular response. These VRFs can be described by apparent molecular weight (MW) and isoelectric point (pI) as provided in Table I.

TABLE I
VRFs Decreased in Blood of Subjects Having Vascular Response

| VRF | pI | MW (Da) |
|---|---|---|
| VRF-1 | 6.9 | 55,862 |
| VRF-2 | 4.5 | 12,427 |
| VRF-3 | 5.9 | 53,798 |
| VRF-4 | 6.6 | 55,660 |
| VRF-5 | 5.4 | 66,982 |
| VRF-6 | 6.3 | 59,720 |
| VRF-7 | 7.5 | 45,196 |
| VRF-8 | 5.0 | 36,628 |
| VRF-9 | 6.8 | 38,561 |
| VRF-10 | 4.6 | 53,996 |
| VRF-11 | 6.4 | 43,053 |
| VRF-12 | 7.0 | 50,908 |
| VRF-13 | 5.3 | 36,415 |
| VRF-14 | 5.1 | 36,546 |
| VRF-15 | 7.6 | 40,071 |
| VRF-16 | 5.9 | 93,545 |
| VRF-17 | 7.2 | 45,318 |
| VRF-18 | 5.5 | 35,251 |
| VRF-19 | 5.4 | 30,705 |
| VRF-20 | 5.8 | 88,662 |
| VRF-21 | 6.4 | 30,462 |
| VRF-22 | 5.6 | 117,048 |
| VRF-23 | 5.3 | 146,763 |
| VRF-24 | 6.9 | 25,788 |
| VRF-25 | 7.5 | 36,779 |
| VRF-26 | 6.4 | 22,672 |
| VRF-27 | 6.4 | 57,270 |
| VRF-28 | 5.0 | 31,998 |
| VRF-29 | 6.1 | 103,294 |
| VRF-30 | 6.3 | 55,313 |
| VRF-31 | 5.6 | 89,343 |
| VRF-32 | 7.5 | 47,734 |
| VRF-34 | 6.3 | 114,395 |
| VRF-35 | 4.7 | 15,304 |
| VRF-36 | 4.9 | 67,894 |
| VRF-37 | 6.7 | 23,985 |
| VRF-38 | 5.9 | 51,173 |
| VRF-40 | 6.6 | 109,011 |
| VRF-42 | 4.6 | 10,631 |
| VRF-131 | 8.6 | 62,760 |
| VRF-132 | 8.6 | 56,316 |
| VRF-133 | 6.1 | 170,804 |
| VRF-134 | 5.5 | 106,240 |
| VRF-135 | 6.4 | 76,184 |
| VRF-136 | 6.6 | 56,091 |
| VRF-137 | 5.3 | 108,420 |
| VRF-138 | 6.2 | 161,240 |
| VRF-139 | 6.3 | 56,572 |
| VRF-140 | 8.4 | 47,387 |
| VRF-141 | 6.0 | 159,928 |
| VRF-142 | 5.3 | 40,795 |
| VRF-143 | 8.0 | 46,894 |
| VRF-144 | 5.2 | 104,122 |
| VRF-145 | 6.2 | 47,376 |
| VRF-146 | 5.5 | 48,580 |
| VRF-147 | 7.2 | 57,090 |
| VRF-148 | 7.4 | 51,981 |
| VRF-149 | 6.9 | 57,970 |
| VRF-150 | 7.3 | 54,641 |
| VRF-151 | 7.2 | 74,287 |
| VRF-152 | 6.7 | 108,339 |
| VRF-153 | 5.9 | 164,081 |
| VRF-183 | 5.2 | 90,873 |
| VRF-184 | 6.8 | 35,881 |
| VRF-185 | 6.9 | 47,790 |
| VRF-186 | 6.3 | 66,687 |
| VRF-187 | 6.3 | 33,460 |
| VRF-188 | 6.6 | 29,779 |
| VRF-226 | 5.3 | 102,172 |
| VRF-227 | 5.5 | 163,669 |
| VRF-228 | 6.3 | 160,221 |
| VRF-229 | 6.4 | 66,374 |
| VRF-246 | 6.4 | 34,560 |

The second group consists of VRFs that are increased in the blood of subjects having vascular response as compared with the blood of subjects free from vascular response. These VRFs can be described by MW and pI as follows:

TABLE II
VRFs Increased in Blood of Subjects Having Vascular Response

| VRF | pI | MW (Da) |
|---|---|---|
| VRF-43 | 5.3 | 15,671 |
| VRF-44 | 5.7 | 42,612 |
| VRF-45 | 6.6 | 65,153 |
| VRF-46 | 4.6 | 26,025 |
| VRF-47 | 5.8 | 63,791 |
| VRF-48 | 7.5 | 41,646 |
| VRF-50 | 6.5 | 28,210 |
| VRF-51 | 6.9 | 46,197 |
| VRF-52 | 7.3 | 14,777 |
| VRF-53 | 5.7 | 24,740 |
| VRF-54 | 6.0 | 54,784 |
| VRF-55 | 4.7 | 15,983 |
| VRF-56 | 5.5 | 46,824 |
| VRF-57 | 5.4 | 41,990 |
| VRF-58 | 4.8 | 42,404 |
| VRF-59 | 4.8 | 49,983 |
| VRF-60 | 6.7 | 65,458 |
| VRF-61 | 5.6 | 41,149 |
| VRF-62 | 6.2 | 80,775 |
| VRF-63 | 6.0 | 89,519 |
| VRF-64 | 5.9 | 55,255 |
| VRF-65 | 5.6 | 23,764 |
| VRF-66 | 6.0 | 52,425 |
| VRF-67 | 7.4 | 12,797 |
| VRF-68 | 5.0 | 32,691 |
| VRF-69 | 6.3 | 52,057 |
| VRF-70 | 4.4 | 35,889 |
| VRF-71 | 7.0 | 72,009 |
| VRF-72 | 5.3 | 46,549 |
| VRF-73 | 5.3 | 56,876 |
| VRF-74 | 4.7 | 31,099 |
| VRF-75 | 5.5 | 51,088 |
| VRF-76 | 5.3 | 41,608 |
| VRF-77 | 5.9 | 106,831 |
| VRF-79 | 4.5 | 28,816 |
| VRF-80 | 5.8 | 109,095 |
| VRF-81 | 6.8 | 27,783 |
| VRF-82 | 5.5 | 44,702 |
| VRF-83 | 4.5 | 35,312 |
| VRF-84 | 5.7 | 110,078 |
| VRF-85 | 5.2 | 118,045 |
| VRF-86 | 5.9 | 65,200 |
| VRF-87 | 6.3 | 53,617 |
| VRF-88 | 7.6 | 47,020 |
| VRF-89 | 4.9 | 39,092 |
| VRF-90 | 7.2 | 46,463 |
| VRF-91 | 4.4 | 36,411 |
| VRF-92 | 5.5 | 64,501 |
| VRF-93 | 4.6 | 71,770 |
| VRF-94 | 5.0 | 49,783 |
| VRF-95 | 5.3 | 23,986 |
| VRF-96 | 7.0 | 56,480 |
| VRF-97 | 6.6 | 51,988 |
| VRF-98 | 4.6 | 79,952 |
| VRF-99 | 4.6 | 70,686 |
| VRF-100 | 7.0 | 53,025 |
| VRF-101 | 5.0 | 88,760 |
| VRF-102 | 5.1 | 75,937 |
| VRF-103 | 7.5 | 27,997 |
| VRF-104 | 4.9 | 45,028 |
| VRF-105 | 4.7 | 30,156 |
| VRF-106 | 5.2 | 23,929 |
| VRF-107 | 6.4 | 103,089 |
| VRF-108 | 6.1 | 94,872 |
| VRF-109 | 6.5 | 44,363 |
| VRF-110 | 6.6 | 117,961 |
| VRF-111 | 5.0 | 12,909 |
| VRF-112 | 6.0 | 47,782 |
| VRF-113 | 6.3 | 24,290 |
| VRF-114 | 5.5 | 62,780 |
| VRF-115 | 5.0 | 35,008 |
| VRF-116 | 7.2 | 50,820 |
| VRF-117 | 7.8 | 55,125 |
| VRF-118 | 5.0 | 61,654 |
| VRF-119 | 5.3 | 36,415 |
| VRF-120 | 4.8 | 67,581 |
| VRF-122 | 5.9 | 18,986 |
| VRF-123 | 6.3 | 49,733 |
| VRF-124 | 4.7 | 68,327 |
| VRF-125 | 5.3 | 27,768 |
| VRF-126 | 6.1 | 57,486 |
| VRF-127 | 6.2 | 54,118 |
| VRF-128 | 7.6 | 24,430 |
| VRF-129 | 5.0 | 52,708 |
| VRF-130 | 7.6 | 52,327 |
| VRF-153 | 5.9 | 164,081 |
| VRF-154 | 5.7 | 93,937 |
| VRF-155 | 5.8 | 98,012 |
| VRF-156 | 5.9 | 46,795 |
| VRF-157 | 5.0 | 71,952 |
| VRF-158 | 5.9 | 38,180 |
| VRF-159 | 5.0 | 86,263 |
| VRF-160 | 4.9 | 57,857 |
| VRF-161 | 5.3 | 61,350 |
| VRF-162 | 5.8 | 92,965 |
| VRF-163 | 4.9 | 42,639 |
| VRF-164 | 6.9 | 41,691 |
| VRF-165 | 4.5 | 17,273 |
| VRF-166 | 5.2 | 53,618 |
| VRF-167 | 5.5 | 15,280 |
| VRF-168 | 5.9 | 22,620 |
| VRF-169 | 5.1 | 21,015 |
| VRF-170 | 5.7 | 25,824 |
| VRF-171 | 4.9 | 73,274 |
| VRF-172 | 7.8 | 26,158 |
| VRF-173 | 5.1 | 64,281 |
| VRF-174 | 5.6 | 12,587 |
| VRF-175 | 5.5 | 13,378 |
| VRF-176 | 6.3 | 12,252 |
| VRF-177 | 4.9 | 94,792 |
| VRF-178 | 5.2 | 63,354 |
| VRF-179 | 4.8 | 46,315 |
| VRF-180 | 6.3 | 80,457 |
| VRF-181 | 7.6 | 11,178 |
| VRF-182 | 8.0 | 10,939 |
| VRF-189 | 7.0 | 26,002 |
| VRF-190 | 5.5 | 26,171 |
| VRF-191 | 4.5 | 34,811 |
| VRF-192 | 7.5 | 77,234 |
| VRF-193 | 5.2 | 44,506 |
| VRF-194 | 7.5 | 26,138 |
| VRF-195 | 5.0 | 64,764 |
| VRF-196 | 4.4 | 85,200 |
| VRF-197 | 5.0 | 113,373 |
| VRF-198 | 6.2 | 102,667 |
| VRF-199 | 4.5 | 31,524 |
| VRF-200 | 4.7 | 28,230 |
| VRF-201 | 4.5 | 57,154 |
| VRF-202 | 5.5 | 56,331 |
| VRF-203 | 5.1 | 79,689 |
| VRF-204 | 4.8 | 73,028 |
| VRF-205 | 4.7 | 76,949 |
| VRF-206 | 7.6 | 91,970 |
| VRF-207 | 4.6 | 21,586 |
| VRF-208 | 4.7 | 59,670 |
| VRF-209 | 7.7 | 76,246 |
| VRF-210 | 7.4 | 70,172 |
| VRF-211 | 7.0 | 23,768 |
| VRF-212 | 5.8 | 166,718 |
| VRF-213 | 4.4 | 86,497 |
| VRF-214 | 9.2 | 56,906 |
| VRF-215 | 4.8 | 23,997 |
| VRF-216 | 7.7 | 70,910 |
| VRF-217 | 4.5 | 47,550 |
| VRF-218 | 7.9 | 90,621 |
| VRF-219 | 6.1 | 127,699 |
| VRF-220 | 5.5 | 172,394 |
| VRF-221 | 4.6 | 50,897 |
| VRF-222 | 4.2 | 12,107 |
| VRF-223 | 8.1 | 71,265 |
| VRF-224 | 4.8 | 41,462 |
| VRF-225 | 4.3 | 20,955 |
| VRF-230 | 5.1 | 55,780 |
| VRF-231 | 5.3 | 30,594 |
| VRF-232 | 6.8 | 86,976 |
| VRF-233 | 5.1 | 53,782 |
| VRF-234 | 6.1 | 12,243 |
| VRF-235 | 5.0 | 34,090 |
| VRF-236 | 5.1 | 24,090 |
| VRF-237 | 5.2 | 41,154 |
| VRF-238 | 4.5 | 60,771 |
| VRF-239 | 5.4 | 89,494 |
| VRF-240 | 7.9 | 34,448 |
| VRF-241 | 4.2 | 45,046 |
| VRF-242 | 8.3 | 46,524 |
| VRF-243 | 4.7 | 129,281 |
| VRF-244 | 6.7 | 84,357 |
| VRF-245 | 6.9 | 87,338 |
| VRF-247 | 6.3 | 33,925 |
| VRF-248 | 6.1 | 27,002 |
| VRF-249 | 7.9 | 77,468 |
| VRF-250 | 4.4 | 70,668 |
| VRF-251 | 4.3 | 10,895 |
| VRF-252 | 4.2 | 41,478 |
| VRF-253 | 6.0 | 56,223 |
| VRF-254 | 7.5 | 40,506 |
| VRF-255 | 8.1 | 52,364 |
| VRF-256 | 5.7 | 83,735 |
| VRF-257 | 6.0 | 12,254 |
| VRF-258 | 4.2 | 12,240 |
| VRF-259 | 6.5 | 23,684 |
| VRF-260 | 6.2 | 60,551 |
| VRF-261 | 6.1 | 23,907 |
| VRF-262 | 4.3 | 43,115 |
| VRF-263 | 4.8 | 66,951 |
| VRF-264 | 4.6 | 97,834 |
| VRF-265 | 7.9 | 52,366 |
| VRF-266 | 8.1 | 105,028 |
| VRF-267 | 4.4 | 39,734 |
| VRF-268 | 4.3 | 42,662 |
| VRF-269 | 5.3 | 90,717 |
| VRF-270 | 4.1 | 56,649 |
| VRF-271 | 8.6 | 81,530 |
| VRF-272 | 4.5 | 53,381 |

For any given VRF, the signal obtained upon analyzing blood from subjects having vascular response relative to the signal obtained upon analyzing blood from subjects free from vascular response will depend upon the particular analytical protocol and detection technique that is used. Accordingly, the present invention contemplates that each laboratory will, based on the present description, establish a reference range for each VRF in subjects free from vascular response according to the analytical protocol and detection technique in use, as is conventional in the diagnostic art. Preferably, at least one control positive blood sample from a subject known to have vascular response or at least one control negative blood sample from a subject known to be free from vascular response (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no discernable protein feature.

In a preferred embodiment, the signal associated with a VRF in the blood of a subject (e,g., a subject suspected of having or known to have vascular response) is normalized with reference to one or more ERFs detected in the same 2D gel. As will be apparent to one of ordinary skill in the art, such ERFs may readily be determined by comparing different samples using the Preferred Technology. Suitable ERFs include (but are not limited to) that describe in the following table.

TABLE III
Expression Reference Features

| ERF# | PI | MW (Da) |
|---|---|---|
| ERF-1 | 4.44 | 48590 |
| ERF-2 | 4.42 | 35889 |

As those of skill in the art will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight and the apparent isoelectric point of a feature or protein isoform as measured in exact accordance with the Reference Protocol identified in Section 6 below. When the Reference Protocol is followed and when samples are run in duplicate or a higher number of replicates, variation in the measured mean pI of a VRF or VRPI is typically less than 3% and variation in the measured mean MW of a VRF or VRPI is typically less than 5%. Where the skilled artisan wishes to deviate from the Reference Protocol, calibration experiments should be performed to compare the MW and pI for each VRF or protein isoform as detected (a) by the Reference Protocol and (b) by the deviant protocol.

VRFs can be used for detection, prognosis, diagnosis, or monitoring of vascular response, or for identifying patients most likely to respond to a specific therapeutic treatment, or for drug development. In one embodiment of the invention, blood from a subject (e.g., a subject treated with a drug candidiate, or suspected of having vascular response) is analyzed by 2D electrophoresis for quantitative detection of one or more of the following VRFs: VRF-1, VRF-2, VRF-3, VRF-4, VRF-5, VRF-6, VRF-7, VRF-8, VRF-9, VRF-10, VRF-11, VRF-12, VRF-13, VRF-14, VRF-15, VRF-16, VRF-17, VRF-18, VRF-19, VRF-20, VRF-21, VRF-22, VRF-23, VRF-24, VRF-25, VRF-26, VRF-27, VRF-28, VRF-29, VRF-30, VRF-31, VRF-32, VRF-34, VRF-35, VRF-36, VRF-37, VRF-38, VRF-40, VRF-42, VRF-131, VRF-132, VRF-133, VRF-134, VRF-135, VRF-136, VRF-137, VRF-138, VRF-139, VRF-140, VRF-141, VRF-142, VRF-143, VRF-144, VRF-145, VRF-146, VRF-147, VRF-148, VRF-149, VRF-150, VRF-151, VRF-152, VRF-153, VRF-183, VRF-184, VRF-185, VRF-186, VRF-187, VRF-188, VRF-226, VRF-227, VRF-228, VRF-229, VRF-246. A decreased abundance of said one or more VRFs in the blood from the subject relative to blood from a subject or subjects free from vascular response (e.g., a control sample or a previously determined reference range) indicates the presence of vascular response.

In another embodiment of the invention, blood from a subject is analyzed by 2D electrophoresis for quantitative detection of one or more of the following VRFs: VRF-43, VRF-44, VRF-45, VRF-46, VRF-47, VRF-48, VRF-50, VRF-51, VRF-52, VRF-53, VRF-54, VRF-55, VRF-56, VRF-57, VRF-58, VRF-59, VRF-60, VRF-61, VRF-62, VRF-63, VRF-64, VRF-65, VRF-66, VRF-67, VRF-68, VRF-69, VRF-70, VRF-71, VRF-72, VRF-73, VRF-74, VRF-75, VRF-76, VRF-77, VRF-79, VRF-80, VRF-81, VRF-82, VRF-83, VRF-84, VRF-85, VRF-86, VRF-87, VRF-88, VRF-89, VRF-90, VRF-91, VRF-92, VRF-93, VRF-94, VRF-95, VRF-96, VRF-97, VRF-98, VRF-99, VRF-100, VRF-101, VRF-102, VRF-103, VRF-104, VRF-105, VRF-106, VRF-107, VRF-108, VRF-109, VRF-110, VRF-111, VRF-112, VRF-113, VRF-114, VRF-115, VRF-116, VRF-117, VRF-118, VRF-119, VRF-120, VRF-122, VRF-123, VRF-124, VRF-125, VRF-126, VRF-127, VRF-128, VRF-129, VRF-130, VRF-153, VRF-154, VRF-155, VRF-156, VRF-157, VRF-158, VRF-159, VRF-160, VRF-161, VRF-162, VRF-163, VRF-164, VRF-165, VRF-166, VRF-167, VRF-168, VRF-169, VRF-170, VRF-171, VRF-172, VRF-173, VRF-174, VRF-175, VRF-176, VRF-177, VRF-178, VRF-179, VRF-180, VRF-181, VRF-182, VRF-189, VRF-l90, VRF-191, VRF-192, VRF-193, VRF-194, VRF-195, VRF-196, VRF-197, VRF-198, VRF-199, VRF-200, VRF-201, VRF-202, VRF-203, VRF-204, VRF-205, VRF-206, VRF-207, VRF-208, VRF-209, VRF-210, VRF-211, VRF-212, VRF-213, VRF-214, VRF-215, VRF-216, VRF-217, VRF-218, VRF-219, VRF-220, VRF-221, VRF-222, VRF-223, VRF-224, VRF-225, VRF-230, VRF-231, VRF-232, VRF-233, VRF-234, VRF-235, VRF-236, VRF-237, VRF-238, VRF-239, VRF-240, VRF-241, VRF-242, VRF-243, VRF-244, VRF-245, VRF-247, VRF-248, VRF-249, VRF-250, VRF-251, VRF-252, VRF-253, VRF-254, VRF-255, VRF-256, VRF-257, VRF-258, VRF-259, VRF-260, VRF-261, VRF-262, VRF-263, VRF-264, VRF-265, VRF-266, VRF-267, VRF-268, VRF-269, VRF-270, VRF-271, VRF-272. An increased abundance of said one or more VRFs in the blood from the subject relative to blood from a subject or subjects free from vascular response (e.g., a control sample or a previously determined reference range) indicates the presence of vascular response.

In yet another embodiment, blood from a subject is analyzed by 2D electrophoresis for quantitative detection of (a) one or more VRFs or any combination of them, whose decreased abundance indicates the presence of vascular response, i.e.; VRF-1, VRF-2, VRF-3, VRF-4, VRF-5, VRF-6, VRF-7, VRF-8, VRF-9, VRF-10, VRF-11, VRF-12, VRF-13, VRF-14, VRF-15, VRF-16, VRF-17, VRF-18, VRF-19, VRF-20, VRF-21, VRF-22, VRF-23, VRF-24, VRF-25, VRF-26, VRF-27, VRF-28, VRF-29, VRF-30, VRF-31, VRF-32, VRF-34, VRF-35, VRF-36, VRF-37, VRF-38, VRF-40, VRF-42, VRF-131, VRF-132, VRF-133, VRF-134, VRF-135, VRF-136, VRF-137, VRF-138, VRF-139, VRF-140, VRF-141, VRF-142, VRF-143, VRF-144, VRF-145, VRF-146, VRF-147, VRF-148, VRF-149, VRF-150, VRF-151, VRF-152, VRF-153, VRF-183, VRF-184, VRF-185, VRF-186, VRF-187, VRF-188, VRF-226, VRF-227, VRF-228, VRF-229, VRF-246, and (b) one or more VRFs or any combination of them, whose increased abundance indicates the presence of vascular response i.e. VRF-43, VRF-44, VRF-45, VRF-46, VRF-47, VRF-48, VRF-50, VRF-51, VRF-52, VRF-53, VRF-54, VRF-55, VRF-56, VRF-57, VRF-58, VRF-59, VRF-60, VRF-61, VRF-62, VRF-63, VRF-64, VRF-65, VRF-66, VRF-67, VRF-68, VRF-69, VRF-70, VRF-71, VRF-72, VRF-73, VRF-74, VRF-75, VRF-76, VRF-77, VRF-79, VRF-80, VRF-81, VRF-82, VRF-83, VRF-84, VRF-85, VRF-86, VRF-87, VRF-88, VRF-89, VRF-90, VRF-91, VRF-92, VRF-93, VRF-94, VRF-95, VRF-96, VRF-97, VRF-98, VRF-99, VRF-100, VRF-101, VRF-102, VRF-103, VRF-104, VRF-105, VRF-106, VRF-107, VRF-108, VRF-109, VRF-110, VRF-111, VRF-112, VRF-113, VRF-114, VRF-115, VRF-116, VRF-117, VRF-118, VRF-119, VRF-120, VRF-122, VRF-123, VRF-124, VRF-125, VRF-126, VRF-127, VRF-128, VRF-129, VRF-130, VRF-153, VRF-154, VRF-155, VRF-156, VRF-157, VRF-158, VRF-159, VRF-160, VRF-161, VRF-162, VRF-163, VRF-164, VRF-165, VRF-166, VRF-167, VRF-168, VRF-169, VRF-170, VRF-171, VRF-172, VRF-173, VRF-174, VRF-175, VRF-176, VRF-177, VRF-178, VRF-179, VRF-180, VRF-181, VRF-182, VRF-189, VRF-190, VRF-191, VRF-192, VRF-193, VRF-194, VRF-195, VRF-196, VRF-197, VRF-198, VRF-199, VRF-200, VRF-201, VRF-202, VRF-203, VRF-204, VRF-205, VRF-206, VRF-207, VRF-208, VRF-209, VRF-210, VRF-211, VRF-212, VRF-213, VRF-214, VRF-215, VRF-216, VRF-217, VRF-218, VRF-219, VRF-220, VRF-221, VRF-222, VRF-223, VRF-224, VRF-225, VRF-230, VRF-231, VRF-232, VRF-233, VRF-234, VRF-235, VRF-236, VRF-237, VRF-238, VRF-239, VRF-240, VRF-241, VRF-242, VRF-243, VRF-244, VRF-245, VRF-247, VRF-248, VRF-249, VRF-250, VRF-251, VRF-252, VRF-253, VRF-254, VRF-255, VRF-256, VRF-257, VRF-258, VRF-259, VRF-260, VRF-261, VRF-262, VRF-263, VRF-264, VRF-265, VRF-266, VRF-267, VRF-268, VRF-269, VRF-270, VRF-271, VRF-272.

In yet another embodiment of the invention, blood from a subject is analyzed by 2D electrophoresis for quantitative detection of one or more of the following, VRF-1, VRF-2, VRF-3, VRF-4, VRF-5, VRF-6, VRF-7, VRF-8, VRF-9, VRF-10, VRF-11, VRF-12, VRF-13, VRF-14, VRF-15, VRF-16, VRF-17, VRF-18, VRF-19, VRF-20, VRF-21, VRF-22, VRF-23, VRF-24, VRF-25, VRF-26, VRF-27, VRF-28, VRF-29, VRF-30, VRF-31, VRF-32, VRF-34, VRF-35, VRF-36, VRF-37, VRF-38, VRF-40, VRF-42, VRF-43, VRF-44, VRF-45, VRF-46, VRF-47, VRF-48, VRF-50, VRF-51, VRF-52, VRF-53, VRF-54, VRF-55, VRF-56, VRF-57, VRF-58, VRF-59, VRF-60, VRF-61, VRF-62, VRF-63, VRF-64, VRF-65, VRF-66, VRF-67, VRF-68, VRF-69, VRF-70, VRF-71, VRF-72, VRF-73, VRF-74, VRF-75, VRF-76, VRF-77, VRF-79, VRF-80, VRF-81, VRF-82, VRF-83, VRF-84, VRF-85, VRF-86, VRF-87, VRF-88, VRF-89, VRF-90, VRF-91, VRF-92, VRF-93, VRF-94, VRF-95, VRF-96, VRF-97, VRF-98, VRF-99, VRF-100, VRF-101, VRF-102, VRF-103, VRF-104, VRF-105, VRF-106, VRF-107, VRF-108, VRF-109, VRF-110, VRF-111, VRF-112, VRF-113, VRF-114, VRF-115, VRF-116, VRF-117, VRF-118, VRF-119, VRF-120, VRF-122, VRF-123, VRF-124, VRF-125, VRF-126, VRF-127, VRF-128, VRF-129, VRF-130, VRF-131, VRF-132, VRF-133, VRF-134, VRF-135, VRF-136, VRF-137, VRF-138, VRF-139, VRF-140, VRF-141, VRF-142, VRF-143, VRF-144, VRF-145, VRF-146, VRF-147, VRF-148, VRF-149, VRF-150, VRF-151, VRF-152, VRF-153, VRF-154, VRF-155, VRF-156, VRF-157, VRF-158, VRF-159, VRF-160, VRF-161, VRF-162, VRF-163, VRF-164, VRF-165, VRF-166, VRF-167, VRF-168, VRF-169, VRF-170, VRF-171, VRF-172, VRF-173, VRF-174, VRF-175, VRF-176, VRF-177, VRF-178, VRF-179, VRF-180, VRF-181, VRF-182, VRF-183, VRF-184, VRF-185, VRF-186, VRF-187, VRF-188, VRF-189, VRF-190, VRF-191, VRF-192, VRF-193, VRF-194, VRF-195, VRF-196, VRF-197, VRF-198, VRF-199, VRF-200, VRF-201, VRF-202, VRF-203, VRF-204, VRF-205, VRF-206, VRF-207, VRF-208, VRF-209, VRF-210, VRF-211, VRF-212, VRF-213, VRF-214, VRF-215, VRF-216, VRF-217, VRF-218, VRF-219, VRF-220, VRF-221, VRF-222, VRF-223, VRF-224, VRF-225, VRF-226, VRF-227, VRF-228, VRF-229, VRF-230, VRF-231, VRF-232, VRF-233, VRF-234, VRF-235, VRF-236, VRF-237, VRF-238, VRF-239, VRF-240, VRF-241, VRF-242, VRF-243, VRF-244, VRF-245, VRF-246, VRF-247, VRF-248, VRF-249, VRF-250, VRF-251, VRF-252, VRF-253, VRF-254, VRF-255, VRF-256, VRF-257, VRF-258, VRF-259, VRF-260, VRF-261, VRF-262, VRF-263, VRF-264, VRF-265, VRF-266, VRF-267, VRF-268, VRF-269, VRF-270, VRF-271, VRF-272, wherein the ratio of the one or more VRFs relative to an Expression Reference Feature (ERF) indicates whether vascular response is present. In a specific embodiment, a decrease in one or more VRF/ERF ratios in a test sample relative to the VRF/ERF ratios in a control sample or a reference range indicates the presence of vascular response; VRF-1, VRF-2, VRF-3, VRF-4, VRF-5, VRF-6, VRF-7, VRF-8, VRF-9, VRF-10, VRF-11, VRF-12, VRF-13, VRF-14, VRF-15, VRF-16, VRF-17, VRF-18, VRF-19, VRF-20, VRF-21, VRF-22, VRF-23, VRF-24, VRF-25, VRF-26, VRF-27, VRF-28, VRF-29, VRF-30, VRF-31, VRF-32, VRF-34, VRF-35, VRF-36, VRF-37, VRF-38, VRF-40, VRF-42, VRF-131, VRF-132, VRF-133, VRF-134, VRF-135, VRF-136, VRF-137, VRF-138, VRF-139, VRF-140, VRF-141, VRF-142, VRF-143, VRF-144, VRF-145, VRF-146, VRF-147, VRF-148, VRF-149, VRF-150, VRF-151, VRF-152, VRF-153, VRF-183, VRF-184, VRF-185, VRF-186, VRF-187, VRF-188, VRF-226, VRF-227, VRF-228, VRF-229, VRF-246, are suitable VRFs for this purpose. In another specific embodiment, an increase in one or more VRF/ERF ratios in a test sample relative to the VRF/ERF ratios in a control sample or a reference range indicates the presence of vascular response; VRF-43, VRF-44, VRF-45, VRF-46, VRF-47, VRF-48, VRF-50, VRF-51, VRF-52, VRF-53, VRF-54, VRF-55, VRF-56, VRF-57, VRF-58, VRF-59, VRF-60, VRF-61, VRF-62, VRF-63, VRF-64, VRF-65, VRF-66, VRF-67, VRF-68, VRF-69, VRF-70, VRF-71, VRF-72, VRF-73, VRF-74, VRF-75, VRF-76, VRF-77, VRF-79, VRF-80, VRF-81, VRF-82, VRF-83, VRF-84, VRF-85, VRF-86, VRF-87, VRF-88, VRF-89, VRF-90, VRF-91, VRF-92, VRF-93, VRF-94, VRF-95, VRF-96, VRF-97, VRF-98, VRF-99, VRF-100, VRF-101, VRF-102, VRF-103, VRF-104, VRF-105, VRF-106, VRF-107, VRF-108, VRF-109, VRF-110, VRF-111, VRF-112, VRF-113, VRF-114, VRF-115, VRF-116, VRF-117, VRF-118, VRF-119, VRF-120, VRF-122, VRF-123, VRF-124, VRF-125, VRF-126, VRF-127, VRF-128, VRF-129, VRF-130, VRF-153, VRF-154, VRF-155, VRF-156, VRF-157, VRF-158, VRF-159, VRF-160, VRF-161, VRF-162, VRF-163, VRF-164, VRF-165, VRF-166, VRF-167, VRF-168, VRF-169, VRF-170, VRF-171, VRF-172, VRF-173, VRF-174, VRF-175, VRF-176, VRF-177, VRF-178, VRF-179, VRF-180, VRF-181, VRF-182, VRF-189, VRF-190, VRF-191, VRF-192, VRF-193, VRF-194, VRF-195, VRF-196, VRF-197, VRF-198, VRF-199, VRF-200, VRF-201, VRF-202, VRF-203, VRF-204, VRF-205, VRF-206, VRF-207, VRF-208, VRF-209, VRF-210, VRF-211, VRF-212, VRF-213, VRF-214, VRF-215, VRF-216, VRF-217, VRF-218, VRF-219, VRF-220, VRF-221, VRF-222, VRF-223, VRF-224, VRF-225, VRF-230, VRF-231, VRF-232, VRF-233, VRF-234, VRF-235, VRF-236, VRF-237, VRF-238, VRF-239, VRF-240, VRF-241, VRF-242, VRF-243, VRF-244, VRF-245, VRF-247, VRF-248, VRF-249, VRF-250, VRF-251, VRF-252, VRF-253, VRF-254, VRF-255, VRF-256, VRF-257, VRF-258, VRF-259, VRF-260, VRF-261, VRF-262, VRF-263, VRF-264, VRF-265, VRF-266, VRF-267, VRF-268, VRF-269, VRF-270, VRF-271, VRF-272, are suitable VRFs for this purpose.

In a further embodiment of the invention, blood from a subject is analyzed by 2D electrophoresis for quantitative detection of (a) one or more VRFs, or any combination of them, whose decreased VRF/ERF ratio(s) in a test sample relative to the VRF/ERF ratio(s) in a control sample indicates the presence of vascular response, i.e.; VRF-1, VRF-2, VRF-3, VRF-4, VRF-5, VRF-6, VRF-7, VRF-8, VRF-9, VRF-10, VRF-11, VRF-12, VRF-13, VRF-14, VRF-15, VRF-16, VRF-17, VRF-18, VRF-19, VRF-20, VRF-21, VRF-22, VRF-23, VRF-24, VRF-25, VRF-26, VRF-27, VRF-28, VRF-29, VRF-30, VRF-31, VRF-32, VRF-34, VRF-35, VRF-36, VRF-37, VRF-38, VRF-40, VRF-42, VRF-131, VRF-132, VRF-133, VRF-134, VRF-135, VRF-136, VRF-137, VRF-138, VRF-139, VRF-140, VRF-141, VRF-142, VRF-143, VRF-144, VRF-145, VRF-146, VRF-147, VRF-148, VRF-149, VRF-150, VRF-151, VRF-152, VRF-153, VRF-183, VRF-184, VRF-185, VRF-186, VRF-187, VRF-188, VRF-226, VRF-227, VRF-228, VRF-229, VRF-246, (b) one or more VRFs, or any combination of them, whose increased VRF/ERF ratio(s) in a test sample relative to the VRF/ERF ratio(s) in a control sample indicates the presence of vascular response, i.e. VRF-43, VRF-44, VRF-45, VRF-46, VRF-47, VRF-48, VRF-50, VRF-51, VRF-52, VRF-53, VRF-54, VRF-55, VRF-56, VRF-57, VRF-58, VRF-59, VRF-60, VRF-61, VRF-62, VRF-63, VRF-64, VRF-65, VRF-66, VRF-67, VRF-68, VRF-69, VRF-70, VRF-71, VRF-72, VRF-73, VRF-74, VRF-75, VRF-76, VRF-77, VRF-79, VRF-80, VRF-81, VRF-82, VRF-83, VRF-84, VRF-85, VRF-86, VRF-87, VRF-88, VRF-89, VRF-90, VRF-91, VRF-92, VRF-93, VRF-94, VRF-95, VRF-96, VRF-97, VRF-98, VRF-99, VRF-100, VRF-101, VRF-102, VRF-103, VRF-104, VRF-105, VRF-106, VRF-107, VRF-108, VRF-109, VRF-110, VRF-111, VRF-112, VRF-113, VRF-114, VRF-115, VRF-116, VRF-117, VRF-118, VRF-119, VRF-120, VRF-122, VRF-123, VRF-124, VRF-125, VRF-126, VRF-127, VRF-128, VRF-129, VRF-130, VRF-153, VRF-154, VRF-155, VRF-156, VRF-157, VRF-158, VRF-159, VRF-160, VRF-161, VRF-162, VRF-163, VRF-164, VRF-165, VRF-166, VRF-167, VRF-168, VRF-169, VRF-170, VRF-171, VRF-172, VRF-173, VRF-174, VRF-175, VRF-176, VRF-177, VRF-178, VRF-179, VRF-180, VRF-181, VRF-182, VRF-189, VRF-190, VRF-191, VRF-192, VRF-193, VRF-194, VRF-195, VRF-196, VRF-197, VRF-198, VRF-199, VRF-200, VRF-201, VRF-202, VRF-203, VRF-204, VRF-205, VRF-206, VRF-207, VRF-208, VRF-209, VRF-210, VRF-211, VRF-212, VRF-213, VRF-214, VRF-215, VRF-216, VRF-217, VRF-218, VRF-219, VRF-220, VRF-221, VRF-222, VRF-223, VRF-224, VRF-225, VRF-230, VRF-231, VRF-232, VRF-233, VRF-234, VRF-235, VRF-236, VRF-237, VRF-238, VRF-239, VRF-240, VRF-241, VRF-242, VRF-243, VRF-244, VRF-245, VRF-247, VRF-248, VRF-249, VRF-250, VRF-251, VRF-252, VRF-253, VRF-254, VRF-255, VRF-256, VRF-257, VRF-258, VRF-259, VRF-260, VRF-261, VRF-262, VRF-263, VRF-264, VRF-265, VRF-266, VRF-267, VRF-268, VRF-269, VRF-270, VRF-271, VRF-272.

In a preferred embodiment, blood from a subject is analyzed for quantitative detection of a plurality of VRFs.

### 5.3 Vascular Response-Associated Protein Isoforms (VRPIs)

In another aspect of the invention, blood from a subject, preferably a living subject, is analyzed for quantitative detection of one or more Vascular Response-Associated Protein Isoforms (VRPIs) for screening or diagnosis of vascular response, to determine the prognosis of a subject having vascular response, to monitor the effectiveness of vascular response therapy, for identifying patients most likely to respond to a particular therapeutic treatment or for drug development and ,in particular, to determine the potential for drug candidates to induce a vascular response. As is well known in the art, a given protein may be expressed as one or more variants (isoforms) that differ in their amino acid composition (e.g. as a result of alternative mRNA or premRNA processing, e.g. alternative splicing or limited proteolysis) or as a result of differential post-translational modification (e.g., glycosylation, phosphorylation, acylation), or both. Thus, proteins of identical amino acid sequence or proteins encoded by a single gene can differ in their pI, MW, or both. It follows that differential presence of a protein isoform does not require differential expression of the gene encoding the protein in question. As used herein, the term “Vascular Response-Associated Protein Isoform” refers to a protein isoform that is differentially present in blood from a subject having vascular response compared with blood from a subject free from vascular response. As used herein, the term “isoform” also refers to a protein that exists in only a single form, i.e., it is not expressed as several variants.

Two groups of VRPIs have been identified by amino acid sequencing of VRFs. VRPIs were isolated, subjected to proteolysis, and analyzed by mass spectrometry using the methods and apparatus of the Preferred Technology. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.com/ and the European Molecular Biology Laboratory web site at www.mann.embl-heidelberg.de/Services/PeptideSearch/. Identification of VRPIs was performed primarily using the SEQUEST search program (Eng et al., 1994, J. Am. Soc. Mass Spectrom. 5:976-989) with raw, uninterpreted tandem mass spectra of tryptic digest peptides as described in the Examples, infra.

The first group consists of VRPIs that are decreased in the blood of subjects having vascular response as compared with the blood of subjects free from vascular response, where the differential presence is significant. The amino acid sequences of tryptic digest peptides of these VRPIs identified by tandem mass spectrometry and database searching as described in the Examples, infra are listed in Table IV in addition to the pIs and MWs of these VRPIs.

TABLE IV
VRPIs Decreased in Blood of Subjects Having Vascular Response

| | | | | Amino Acid Sequences | |
|---|---|---|---|---|---|
| | | | | from Tryptic Digest | Sequence ID |
| VRF | VRPI | pI | MW (Da) | Peptides | (SEQ ID) |
| VRF-1 | VRPI-1 | 6.9 | 55,862 | GLIDEANQDFTNR | SEQ ID 27 |
| | | | | NIMEYLR | SEQ ID 48 |
| VRF-14 | VRPI-14 | 5.1 | 36,546 | DWVQETMAK | SEQ ID 13 |
| | | | | GSFPWQAK | SEQ ID 29 |
| | | | | MGYVSGWGR | SEQ ID 42 |
| | | | | YVMLPVADQEK | SEQ ID 80 |
| VRF-18 | VRPI-18 | 5.5 | 35,251 | GSFPWQAK | SEQ ID 29 |
| | | | | MGYVSGWGR | SEQ ID 42 |
| VRF-19 | VRPI-19 | 5.4 | 30,705 | TANLGAGAAQPLR | SEQ ID 62 |
| | | | | FWDYLR | SEQ ID 26 |
| | | | | LGPLVEQGR | SEQ ID 39 |
| VRF-23 | VRPI-23 | 5.3 | 146,763 | EYTDDSFTNR | SEQ ID 20 |
| | | | | SSTVAPTLPGEVR | SEQ ID 60 |
| | | | | VFFEQGATR | SEQ ID 70 |
| | | | | GQFEDVTLYQGER | SEQ ID 28 |
| VRF-28 | VRPI-28.1 | 5.0 | 31,998 | MTLDDFR | SEQ ID 44 |
| VRF-28 | VRPI-28.2 | 5.0 | 31,998 | ASGIIDTLFQDR | SEQ ID 7 |
| VRF-32 | VRPI-32.1 | 7.5 | 47,734 | ALEESNYELEGK | SEQ ID 5 |
| VRF-32 | VRPI-32.2 | 7.5 | 47,734 | SVSELPIVHR | SEQ ID 61 |
| VRF-36 | VRPI-36 | 4.9 | 67,894 | LGNINTYADDLQNK | SEQ ID 38 |
| | | | | QLDQQVEVFR | SEQ ID 52 |
| VRF-38 | VRPI-38 | 5.9 | 51,173 | YEELQQTAGR | SEQ ID 76 |
| VRF-183 | VRPI-183 | 5.2 | 90,873 | TTDAEFHTFFDER | SEQ ID 68 |
| | | | | VTGWGNLR | SEQ ID 73 |
| | | | | ELLDSYIDGR | SEQ ID 16 |
| | | | | YQNFDPEVK | SEQ ID 78 |

The second group comprises VRPIs that are increased in the blood of subjects having vascular response as compared with the blood of subjects free from vascular response, where the differential presence is significant. The amino acid sequences of tryptic digest peptides of these VRPIs identified by tandem mass spectrometry and database searching are listed in Table V in addition to the pIs and MWs of these VRPIs.

TABLE V
VRPIs Increased in Blood of Subjects Having Vascular Response

| | | | | Amino Acid Sequences | |
|---|---|---|---|---|---|
| | | | | from Tryptic Digest | Sequence ID |
| VRF | VRPI | pI | MW (Da) | Peptides | (SEQ ID) |
| VRF-43 | VRPI-43 | 5.3 | 15,671 | ELYLVAYK | SEQ ID 17 |
| | | | | NGETFQAMVLYGR | SEQ ID 46 |
| VRF-47 | VRPI-47.1 | 5.8 | 63,791 | APQVSTPTLVEAAR | SEQ ID 6 |
| | | | | LGEYGFQNAVLVR | SEQ ID 37 |
| | | | | LVQEVTDFAK | SEQ ID 1 |
| VRF-47 | VRPI-47.2 | 5.8 | 63,791 | FNPVTGEVPPR | SEQ ID 23 |
| VRF-58 | VRPI-58 | 4.8 | 42,404 | ATIDQNLEDLR | SEQ ID 8 |
| | | | | LGNINTYADDLQNK | SEQ ID 38 |
| | | | | QLDQQVEVFR | SEQ ID 52 |
| VRF-61 | VRPI-61 | 5.6 | 41,149 | LVQEVTDFAK | SEQ ID 40 |
| | | | | SIHTLFGDK | SEQ ID 59 |
| VRF-63 | VRPI-63 | 6.0 | 89,519 | FPNAEFAEITK | SEQ ID 24 |
| | | | | APQVSTPTLVEAAR | SEQ ID 6 |
| VRF-65 | VRPI-65 | 5.6 | 23,764 | NHEEEMLALR | SEQ ID 47 |
| | | | | TRLEQEIATYR | SEQ ID 67 |
| VRF-68 | VRPI-68.1 | 5.0 | 32,691 | DRLEEVR | SEQ ID 12 |
| | | | | LGPLVEQGR | SEQ ID 39 |
| | | | | NEVNTMLGQSTEELR | SEQ ID 45 |
| | | | | TANLGAGAAQPLR | SEQ ID 62 |
| VRF-68 | VRPI-68.2 | 5.0 | 32,691 | ASGIIDTLFQDR | SEQ ID 7 |
| | | | | QQSQVLDAMQDSFTR | SEQ ID 55 |
| VRF-72 | VRPI-72 | 5.3 | 46,549 | QNEGFSLTAK | SEQ ID 54 |
| VRF-73 | VRPI-73 | 5.3 | 56,876 | ATVLYQGQR | SEQ ID 9 |
| VRF-75 | VRPI-75 | 5.5 | 51,088 | QLSLLTTMSNR | SEQ ID 53 |
| | | | | RTQVPEVFLSK | SEQ ID 56 |
| | | | | TQVPEVFLSK | SEQ ID 66 |
| VRF-86 | VRPI-86 | 5.9 | 65,200 | FNPVTGEVPPR | SEQ ID 23 |
| | | | | NPVTSVDAAFR | SEQ ID 50 |
| VRF-93 | VRPI-93 | 4.6 | 71,770 | EDFPFLR | SEQ ID 14 |
| | | | | YVIEFIAR | SEQ ID 79 |
| VRF-94 | VRPI-94 | 5.0 | 49,783 | LAAAVSNFGYDLYR | SEQ ID 36 |
| VRF-95 | VRPI-95 | 5.3 | 23,986 | FGLYSDQMR | SEQ ID 22 |
| | | | | WNEEVEAYR | SEQ ID 74 |
| VRF-100 | VRPI-100 | 7.0 | 53,025 | EDGGGWWYNR | SEQ ID 15 |
| | | | | TENGGWTVIQNR | SEQ ID 63 |
| VRF-102 | VRPI-102 | 5.1 | 75,937 | ATWSGAVLAGR | SEQ ID 10 |
| | | | | HQFLLTGDTQGR | SEQ ID 32 |
| VRF-104 | VRPI-104 | 4.9 | 45,028 | ATIDQNLEDLR | SEQ ID 8 |
| | | | | LGNINTYADDLQNK | SEQ ID 38 |
| | | | | QLDQQVEVFR | SEQ ID 52 |
| VRF-106 | VRPI-106 | 5.2 | 23,929 | AKPALEDLR | SEQ ID 4 |
| | | | | DEPPQSPWDR | SEQ ID 11 |
| | | | | THLAPYSDELR | SEQ ID 64 |
| | | | | VQPYLDDFQK | SEQ ID 72 |
| | | | | WQEEMELYR | SEQ ID 75 |
| VRF-108 | VRPI-108 | 6.1 | 94,872 | HVVPNEVVVQR | SEQ ID 33 |
| | | | | AGALNSNDAFVLK | SEQ ID 3 |
| | | | | TPSAAYLWVGTGASEAEK | SEQ ID 65 |
| VRF-115 | VRPI-115 | 5.0 | 35,008 | GWFEPLVEDMQR | SEQ ID 31 |
| | | | | LGPLVEQGR | SEQ ID 39 |
| | | | | NEVNTMLGQSTEELR | SEQ ID 45 |
| | | | | TANLGAGAAQPLR | SEQ ID 62 |
| VRF-116 | VRPI-116 | 7.2 | 50,820 | ADTGTTSEFIDEGAGIR | SEQ ID 2 |
| VRF-118 | VRPI-118.1 | 5.0 | 61,654 | SDVDEDIIPEEDIISR | SEQ ID 57 |
| VRF-118 | VRPI-118.2 | 5.0 | 61,654 | FATNFYQHLADSK | SEQ ID 21 |
| VRF-120 | VRPI-120 | 4.8 | 67,581 | NIPVDSPELK | SEQ ID 49 |
| | | | | YVIEFIAR | SEQ ID 79 |
| | | | | YNAELESGNQFLLYR | SEQ ID 77 |
| VRF-130 | VRPI-130 | 7.6 | 52,327 | EDGGGWWYNR | SEQ ID 15 |
| | | | | TENGGWTVIQNR | SEQ ID 63 |
| VRF-216 | VRPI-216 | 7.7 | 70,910 | EVVADSVWVDVK | SEQ ID 19 |
| VRF-238 | VRPI-238 | 4.5 | 60,771 | FSISTDYSLK | SEQ ID 25 |
| | | | | VFSQQADLSR | SEQ ID 71 |
| | | | | EQPILSEFQEK | SEQ ID 18 |
| | | | | MQQVESSLQPETLK | SEQ ID 43 |
| | | | | IAELFSDLEER | SEQ ID 34 |
| VRF-245 | VRPI-245 | 6.9 | 87,338 | SGYTFQLLR | SEQ ID 58 |
| | | | | QIGYVYR | SEQ ID 51 |
| VRF-249 | VRPI-249 | 7.9 | 77,468 | IPSHAVVAR | SEQ ID 35 |
| | | | | GTDFQLNQLQGK | SEQ ID 30 |
| | | | | VAQEHFGK | SEQ ID 69 |
| | | | | LYLGHSYVTAIR | SEQ ID 41 |
| VRF-269 | VRPI-269 | 5.3 | 90,717 | TTDAEFHTFFDER | SEQ ID 68 |
| | | | | ELLDSYIDGR | SEQ ID 16 |
| | | | | YQNFDPEVK | SEQ ID 78 |

As will be evident to one of skill in the art, based upon the present description, a given VRPI can be described according to the data provided for that VRPI in Table IV or V. The VRPI is a protein comprising a peptide sequence described for that VRPI (preferably comprising a plurality of, more preferably all of, the peptide sequences described for that VRPI) and has a pI of about the value stated for that VRPI (preferably within 10%, more preferably within 5% still more preferably within 1% of the stated value) and has a MW of about the value stated for that VRPI (preferably within 10%, more preferably within 5%, still more preferably within 1% of the stated value).

In one embodiment, blood from a subject is analyzed for quantitative detection of one or more of the following VRPIs: VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-183, or any combination of them, wherein a decreased abundance of the VRPI or VRPIs (or any combination of them) in the blood from the subject relative to blood from a subject or subjects free from vascular response (e.g., a control sample or a previously determined reference range) indicates the presence of vascular response.

In another embodiment of the invention, blood from a subject is analyzed for quantitative detection of one or more of the following VRPIs: VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-216, VRPI-238, VRPI-245, VRPI-249, VRPI-269, or any combination of them, wherein an increased abundance of the VRPI or VRPIs (or any combination of them) in blood from the subject relative to blood from a subject or subjects free from vascular response (e.g., a control sample or a previously determined reference range) indicates the presence of vascular response.

In a further embodiment, blood from a subject is analyzed for quantitative detection of (a) one or more VRPIs, or any combination of them, whose decreased abundance indicates the presence of vascular response, i.e. VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-183, and (b) one or more VRPIs, or any combination of them, whose increased abundance indicates the presence of vascular response, i.e. VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-216, VRPI-238, VRPI-245, VRPI-249, VRPI-269.

In yet a further embodiment, blood from a subject is analysed for quantitative detection of one or more VRPIs and one or more previously known biomarkers of vascular response (e.g., apolipoprotein A1). In accordance with this embodiment, the abundance of each VRPI and known biomarker relative to a control or reference range indicates whether a subject has vascular response.

Preferably, the abundance of a VRPI is normalized to an Expression Reference Protein Isoform (ERPI). ERPIs can be identified by partial amino acid sequencing of ERFs, which are described above, using the methods and apparatus of the Preferred Technology.

As shown above, the VRPIs described herein include previously unknown proteins, as well as isoforms of known proteins where the isoforms were not previously known to be associated with vascular response. For each VRPI, the present invention additionally provides: (a) a preparation comprising the isolated VRPI; (b) a preparation comprising one or more fragments of the VRPI; and (c) antibodies that bind to said VRPI, to said fragments, or both to said VRPI and to said fragments. As used herein, a VRPI is “isolated” when it is present in a preparation that is substantially free of contaminating proteins, i.e., a preparation in which less than 10% (preferably less than 5%, more preferably less than 1%) of the total protein present is contaminating protein(s). A contaminating protein is a protein or protein isoform having a significantly different pI or MW from those of the isolated VRPI, as determined by 2D electrophoresis. As used herein, a “significantly different” pI or MW is one that permits the contaminating protein to be resolved from the VRPI on 2D electrophoresis, performed according to the Reference Protocol.

In one embodiment, an isolated protein is provided, said protein comprising a peptide with the amino acid sequence identified in Table IV or V for a VRPI, said protein having a pI and MW within 10% (preferably within 5%, more preferably within 1%) of the values identified in Table IV or V for that VRPI.

The VRPIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the VRPIs are separated on a 2-D gel by virtue of their MWs and pIs and visualized by staining the gel. In one embodiment, the VRPIs are stained with a fluorescent dye and imaged with a fluorescence scanner. Sypro Red (Molecular Probes, Inc., Eugene, Oregon) is a suitable dye for this purpose. A preferred fluorescent dye is Pyridinium, 4-[2-[4-(dipentylamino)-2-trifluoromethylphenyl] ethenyl]-1-(sulfobutyl)-, inner salt. See U.S. application Ser. No. 09/412,168, filed on Oct. 5, 1999, which is incorporated herein by reference in its entirety.

Alternatively, VRPIs can be detected in an immunoassay. In one embodiment, an immunoassay is performed by contacting a sample from a subject to be tested with an anti-VRPI antibody under conditions such that immunospecific binding can occur if the VRPI is present, and detecting or measuring the amount of any immunospecific binding by the antibody. Preferably, the anti-VRPI antibody preferentially binds to the VRPI rather than to other isoforms of the same protein. Anti-VRPI antibodies can be produced by the methods and techniques described herein; examples of such antibodies known in the art have been reported to recognize a protein having an amino acid sequence corresponding to a sequence of a VRPI, or which have been reported to recognize a protein named in the database selected by searching with the VRPI sequence, are set forth in Table VI. These antibodies shown in Table VI are already reported to bind to the protein of which the VRPI is itself predicted to be a family member.

TABLE VI
Known Antibodies That Recognise VRPIs or VRPI-Related
Polypeptides.

| VRF | VRPI | Antibody | Manufacturer | Catalogue Number |
|---|---|---|---|---|
| VRF-47 | VRPI-47.1 | Chicken anti- | ACCURATE CHEM- | IMS-01-026-02 |
| VRF-61 | VRPI-61 | Albumin, Human | ICAL & SCIENTIFIC | |
| VRF-63 | VRPI-63 | | CORPORATION | |
| VRF-102 | VRPI-102 | Alpha-1-Acid | ACCURATE CHEM- | BYA-6189-1 |
| | | Glycoprotein, | ICAL & SCIENTIFIC | |
| | | Clone: AGP-47 | CORPORATION | |
| | | Mab anti-Human | | |
| VRF-32 | VRPI-32.1 | Anti-Cytokeratin | RDI RESEARCH | RDI-CBL196 |
| | | Type 10 | DIAGNOSTICS, INC | |
| VRF-118 | VRPI-118.2 | Antithrombin III, | ACCURATE CHEM- | BYA-9009-1 |
| | | Clone: BL-ATIII/3, | ICAL & SCIENTIFIC | |
| | | Mab anti-Human | CORPORATION | |
| VRF-36 | VRPI-36 | Apolipoprotein A1 | ACCURATE CHEM- | ACL-20075AP |
| VRF-58 | VRPI-58 | (HDL), Sheep anti- | ICAL & SCIENTIFIC | |
| VRF-95 | VRPI-95 | Human | CORPORATION | |
| VRF-104 | VRPI-104 | | | |
| VRF-106 | VRPI-106 | | | |
| VRF-19 | VRPI-19 | Apolipoprotein E, | ACCURATE CHEM- | YM-5029 |
| VRF-68 | VRPI-68.1 | LDL, VLDL, Clone: | ICAL & SCIENTIFIC | |
| VRF-115 | VRPI-115 | 3D12, Mab anti- | CORPORATION | |
| | | Human, | | |
| | | frozen/paraffin | | |
| VRF-72 | VRPI-72 | C3 Complement, | ACCURATE CHEM- | IMS-01-001-02 |
| VRF-118 | VRPI-118.1 | Chicken anti-Human | ICAL & SCIENTIFIC | |
| VRF-216 | VRPI-216 | | CORPORATION | |
| VRF-38 | VRPI-38 | Cytokeratin 5, 6 & 18, | ACCURATE CHEM- | BMD-V1073 |
| | | 56 & 45kD, Clone: | ICAL & SCIENTIFIC | |
| | | LP34, Mab anti- | CORPORATION | |
| | | Human | | |
| VRF-100 | VRPI-100 | Fibrinogen, Fibrin I, | ACCURATE CHEM- | NYB-18C6 |
| VRF-130 | VRPI-130 | B-beta chain (Bβ 1- | ICAL & SCIENTIFIC | |
| | | 42), Clone: 18C6, | CORPORATION | |
| | | Mab anti-Human | | |
| VRF-108 | VRPI-108 | Gelsolin, plasma + | ACCURATE CHEM- | YBG-4628-6210 |
| | | cytoplasmic, Sheep | ICAL & SCIENTIFIC | |
| | | anti- | CORPORATION | |
| VRF-28 | VRPI-28.2 | Goat anti-Clusterin | RDI RESEARCH | RDI-CLUSTRCabG |
| VRF-68 | VRPI-68.2 | (human) | DIAGNOSTICS, INC | |
| VRF-47 | VRPI-47.2 | Hemopexin, Beta-1, | ACCURATE CHEM- | YN-RHHPX |
| VRF-86 | VRPI-86 | Rabbit anti-Human, | ICAL & SCIENTIFIC | |
| VRF-93 | VRPI-93 | precipitating | CORPORATION | |
| VRF-120 | VRPI-120 | | | |
| VRF-28 | VRPI-28.1 | Monoclonal Anti- | BIODESIGN | M42107M |
| VRF-1 | VRPI-1 | Cytokeratin | INTERNATIONAL | |
| VRF-116 | VRPI-116 | | | |
| VRF-94 | VRPI-94 | Mouse anti-Human | Chemicon International | MAB1059 |
| | | pigment epithelium- | | |
| | | derived factor (PEDF) | | |
| | | (monoclonal) Clone | | |
| | | 10F12.2 | | |
| VRF-183 | VRPI-183 | Prothrombin, Rabbit | ACCURATE CHEM- | AXL-448/2 |
| VRF-269 | VRPI-269 | anti-Human | ICAL & SCIENTIFIC | |
| | | | CORPORATION | |
| VRF-23 | VRPI-23 | Rabbit Polyclonal | DAKO CORPORATION | A0031 |
| | | Anti-Human | | |
| | | Ceruloplasmin | | |

In a particular embodiment, the anti-VRPI antibody binds to the VRPI with at least 2-fold greater affinity, more preferably at least 5-fold greater affinity, still more preferably at least 10-fold greater affinity, than to said other isoforms of the same protein. The skilled artisan can readily assess and determine the ability of the noted antibody to recognize or bind to the VRPI and the specificity of such binding or recognition. When the antibodies in Table VI do not display the required preferential selectivity for the target VRPI, one skilled in the art can generate additional antibodies by using the VRPI itself for the generation of such antibodies.

VRPIs can be transferred from the gel to a suitable membrane (e.g. a PVDF membrane) and subsequently probed in suitable assays that include, without limitation, competitive and non-competitive assay systems using techniques such as western blots and “sandwich” immunoassays using anti-VRPI antibodies as described herein e.g. the antibodies identified in Table VI, or others raised against the VRPIs of interest as those skilled in the art will appreciate based on the present description. The immunoblots can be used to identify those anti-VRPI antibodies displaying the selectivity required to immuno-specifically differentiate a VRPI from other isoforms encoded by the same gene. For example one skilled in the art can identify anti-VRPI antibodies in catalogues of commercially available antibodies. Some examples of companies that supply antibodies include ACCURATE CHEMICAL & SCIENTIFIC CORPORATION http://www.accuratechemical.com; RDI RESEARCH DIAGNOSTICS, INC- http://www.researchd.com/); BIODESIGN INTERNATIONAL- http://www.biodesign.com/; Chemicon International-www.chemicon.com; DAKO CORPORATION www.dako.com.

In one embodiment, binding of antibody in tissue sections can be used to detect aberrant VRPI localization or an aberrant level of one or more VRPIs. In a specific embodiment, antibody to a VRPI can be used to assay a tissue sample (e.g., a vascular biopsy) from a subject for the level of the VRPI where an aberrant level of VRPI is indicative of vascular response. As used herein, an “aberrant level” means a level that is increased or decreased compared with the level in a subject free from vascular response or a reference level. If desired, the comparison can be performed with a matched sample from the same subject, taken from a portion of the body not affected by vascular response.

Any suitable immunoassay can be used, including, without limitation, competitive and non-competitive assay systems using techniques such as western blots, radjoimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a VRPI can be detected in a fluid sample (e.g., spinal fluid, blood or plasma) by means of a two-step sandwich assay. In the first step, a capture reagent (e.g., an anti-VRPI antibody) is used to capture the VRPI. Examples of such antibodies known in the art can be identified as described infra. The capture reagent can optionally be immobilized on a solid phase. In the second step, a directly or indirectly labeled detection reagent is used to detect the captured VRPI. In one embodiment, the detection reagent is a lectin. Any lectin can be used for this purpose that preferentially binds to the VRPI rather than to other isoforms that have the same core protein as the VRPI or to other proteins that share the antigenic determinant recognized by the antibody. In a preferred embodiment, the chosen lectin binds to the VRPI with at least 2-fold greater affinity, more preferably at least 5-fold greater affinity, still more preferably at least 10-fold greater affinity, than to said other isoforms that have the same core protein as the VRPI or to said other proteins that share the antigenic determinant recognized by the antibody. Based on the present description, a lectin that is suitable for detecting a given VRPI can readily be identified by methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the VRPI in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding a VRPI, a related gene, or related nucleic acid sequences or subsequences, including complementary sequences, can also be used in hybridization assays. A nucleotide encoding a VRPI, or subsequences thereof comprising at least 8 nucleotides, preferably at least 12 nucleotides, and most preferably at least 15 nucleotides can be used as a hybridization probe. Hybridization assays can be used for detection, prognosis, diagnosis, or monitoring of conditions, disorders, or disease states, associated with aberrant expression of genes encoding VRPIs, or for differential diagnosis of subjects with signs or symptoms suggestive of vascular response. In particular, such a hybridization assay can be carried out by a method comprising contacting a subject's sample containing nucleic acid with a nucleic acid probe capable of hybridizing to a DNA or RNA that encodes a VRPI, under conditions such that hybridization can occur, and detecting or measuring any resulting hybridization. Nucleotides can be used for therapy of subjects having vascular response, as described below.

The invention also provides diagnostic kits, comprising an anti-VRPI antibody. In addition, such a kit may optionally comprise one or more of the following: (1) instructions for using the anti-VRPI antibody for diagnosis, prognosis, therapeutic monitoring or any combination of these applications; (2) a labeled binding partner to the antibody; (3) a solid phase (such as a reagent strip) upon which the anti-VRPI antibody is immobilized; and (4) a label or insert indicating regulatory approval for diagnostic, prognostic or therapeutic use or any combination thereof. If no labeled binding partner to the antibody is provided, the anti-VRPI antibody itself can be labeled with a detectable marker, e.g., a chemiluminescent, enzymatic, fluorescent, or radioactive moiety.

The invention also provides a kit comprising a nucleic acid probe capable of hybridizing to RNA encoding a VRPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a VRPI, such as by polymerase chain reaction (see, e.g., Innis et al., 1990, PCR Protocols, Academic Press, Inc., San Diego, Calif.), ligase chain reaction (see EP 320,308) use of Q replicase, cyclic probe reaction, or other methods known in the art.

Kits are also provided which allow for the detection of a plurality of VRPIs or a plurality of nucleic acids each encoding a VRPI. A kit can optionally further comprise a predetermined amount of an isolated VRPI protein or a nucleic acid encoding a VRPI, e.g., for use as a standard or control.

### 5.4 Statistical Techniques for Identifying VRPIs and VRPI Clusters

The uni-variate differential analysis tools, such as fold changes, wilcoxon rank sum test and t-test, are useful in identifying individual VRFs or VRPIs that are diagnostically associated with vascular response or in identifying individual VRPIs that regulate the disease process. In most cases, however, those skilled in the art appreciate that the disease process is associated with a combination of VRFs or VRPIs (and to be regulated by a combination of VRPIs), rather than individual VRFs and VRPIs in isolation. The strategies for discovering such combinations of VRFs and VRPIs differ from those for discovering individual VRFs and VRPIs. In such cases, each individual VRF and VRPI can be regarded as one variable and the disease can be regarded as a joint, multi-variate effect caused by interaction of these variables.

The following steps can be used to identify markers from data produced by the Preferred Technology.

The first step is to identify a collection of VRFs or VRPIs that individually show significant association with vascular response. The association between the identified VRFs or VRPIs and vascular response need not be as highly significant as is desirable when an individual VRF or VRPI is used as a diagnostic. Any of the tests discussed above (fold changes, wilcoxon rank sum test, etc.) can be used at this stage. Once a suitable collection of VRFs or VRPIs has been identified, a sophisticated multi-variate analysis capable of identifying clusters can then be used to estimate the significant multivariate associations with vascular response.

Linear Discriminant Analysis (LDA) is one such procedure, which can be used to detect significant association between a cluster of variables (i.e., VRFs or VRPIs) and vascular response. In performing LDA, a set of weights is associated with each variable (i.e., VRF or VRPI) so that the linear combination of weights and the measured values of the variables can identify the disease state by discriminating between subjects having vascular response and subjects free from vascular response. Enhancements to the LDA allow stepwise inclusion (or removal) of variables to optimize the discriminant power of the model. The result of the LDA is therefore a cluster of VRFs or VRPIs which can be used, without limitation, for diagnosis, prognosis, therapy or drug development. Other enhanced variations of LDA, such as Flexible Discriminant Analysis permit the use of non-linear combinations of variables to discriminate a disease state from a normal state. The results of the discriminant analysis can be verified by post-hoc tests and also by repeating the analysis using alternative techniques such as classification trees.

A further category of VRFs or VRPIs can be identified by qualitative measures by comparing the percentage feature presence of a VRF or VRPI of one group of samples (e.g., samples from diseased subjects) with the percentage feature presence of a VRF or VRPI in another group of samples (e.g., samples from control subjects). The “percentage feature presence” of a VRF or VRPI is the percentage of samples in a group of samples in which the VRF or VRPI is detectable by the detection method of choice. For example, if a VRF is detectable in 95 percent of samples from diseased subjects, the percentage feature presence of that VRF in that sample group is 95 percent. If only 5 percent of samples from non-diseased subjects have detectable levels of the same VRF, detection of that VRF in the sample of a subject would suggest that it is likely that the subject suffers from vascular response.

### 5.5 Use in Clinical Studies

The diagnostic methods and compositions of the present invention can assist in monitoring a clinical study, e.g. to evaluate drugs for therapy of vascular response. In one embodiment, candidate molecules are tested for their ability to restore VRF or VRPI levels in a subject having vascular response to levels found in subjects free from vascular response or, in a treated subject (e.g. after treatment with a vasotoxic agent), to preserve VRF or VRPI levels at or near non-vascular response values. The levels of one or more VRFs or VRPIs can be assayed.

In another embodiment, the methods and compositions of the present invention are used to screen candidates for a clinical study to identify individuals having vascular response; such individuals can then be either excluded from or included in the study or can be placed in a separate cohort for treatment or analysis. If desired, the candidates can concurrently be screened to identify individuals with other measures of vascular response (e.g. angiography); procedures for these screens are well known in the art.

### 5.6 Purification Of VRPIs

In particular aspects, the invention provides isolated mammalian VRPIs, preferably rat or human VRPIs, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a fill-length (wild-type) VRPI, e.g., binding to a VRPI substrate or VRPI binding partner, antigenicity (binding to an anti-VRPI antibody), immunogenicity, enzymatic activity and the like.

In specific embodiments, the invention provides fragments of a VRPI comprising at least 5 amino acids, at least 10 amino acids, at least 50 amino acids, or at least 75 amino acids. Fragments lacking some or all of the regions of a VRPI are also provided, as are proteins (e.g., fusion proteins) comprising such fragments. Nucleic acids encoding the foregoing are provided.

Once a recombinant nucleic acid which encodes the VRPI, a portion of the VRPI, or a precursor of the VRPI is identified, the gene product can be analyzed. This is achieved by assays based on the physical or functional properties of the product, including radioactive labeling of the product followed by analysis by gel electrophoresis, immunoassay, etc.

The VRPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the VRPI is identified, the entire amino acid sequence of the VRPI can be deduced from the nucleotide sequence of the gene coding region contained in the recombinant nucleic acid. As a result, the protein can be synthesized by standard chemical methods known in the art (e.g., see Hunkapiller et al., 1984, Nature 310:105-111).

In another alternative embodiment, native VRPIs can be purified from natural sources, by standard methods such as those described above (e.g., immunoaffinity purification).

In a preferred embodiment, VRPIs are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated VRPI that can be recovered from the gel. When used in this way for preparative-scale runs, the Preferred Technology typically provides up to 100 ng, and can provide up to 1000 ng, of an isolated VRPI in a single run. Those of skill in the art will appreciate that a zoom gel can be used in any separation strategy which employs gel isoelectric focusing.

The invention thus provides an isolated VRPI, an isolated VRPI-related polypeptide, and an isolated derivative or fragment of a VRPI or a VRPI-related polypeptide; any of the foregoing can be produced by recombinant DNA techniques or by chemical synthetic methods.

### 5.7 Isolation of DNA Encoding a VRPI

Specific embodiments for the cloning of a gene encoding a VRPI, are presented below by way of example and not of limitation.

The nucleotide sequences of the present invention, including DNA and RNA, and comprising a sequence encoding a VRPI or a fragment thereof, or a VRPI-related polypeptide, may be synthesized using methods known in the art, such as using conventional chemical approaches or polymerase chain reaction (PCR) amplification. The nucleotide sequences of the present invention also permit the identification and cloning of the gene encoding a VRPI homolog or VRPI ortholog including, for example, by screening cDNA libraries, genomic libraries or expression libraries.

For example, to clone a gene encoding a VRPI by PCR techniques, anchored degenerate oligonucleotides (or a set of most likely oligonucleotides) can be designed for all VRPI peptide fragments identified as part of the same protein. PCR reactions under a variety of conditions can be performed with relevant cDNA and genomic DNAs (e.g., from vascular tissue or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):39-42; Dyer K D, Biotechniques, 1995, 19(4):550-2). Vectorette PCR may pe performed with probes that are, for example, anchored degenerate oligonucleotides (or most likely oligonucleotides) coding for VRPI peptide fragments, using as a template a genomic library or cDNA library pools.

Anchored degenerate oligonucleotides (and most likely oligonucleotides) can be designed for all VRPI peptide fragments. These oligonucleotides may be labelled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding a VRPI or VRPI fragment of the present invention are useful for their ability to hybridize selectively with complementary stretches of genes encoding other proteins. Depending on the application, a variety of hybridization conditions may be employed to obtain nucleotide sequences at least 30%, 35%, 40%, 45%, 50%, 55%, 60%, 65%, 70%, 75%, 80%, 85%, 90%, 95% or 99% identical, or 100% identical, to the sequence of a nucleotide encoding a VRPI.

For a high degree of selectivity, relatively stringent conditions are used to form the duplexes, such as low salt or high temperature conditions. As used herein, “highly stringent conditions” means hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2×SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding a VRPI, 37° C. for 90 to 95% identity and 32° C. for 70 to 90% identity.

In the preparation of genomic libraries, DNA fragments are generated, some of which will encode parts or the whole of a VRPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T₄, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U. K. Vol. I, II; Ausubel F. M. et al., eds., 1989, Current Protocols in Molecular Biology, Vol. I, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

Based on the present description, the genomic libraries may be screened with labeled degenerate oligonucleotide probes corresponding to the amino acid sequence of any peptide of the VRPI using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

In Tables IV and V above, some VRPIs disclosed herein were found to correspond to isoforms of previously identified proteins encoded by genes whose sequences are publicly known. To screen such a gene, any probe may be used that is complementary to the gene or its complement; preferably the probe is 10 nucleotides or longer, more preferably 15 nucleotides or longer.

The SWISS-PROT and trEMBL databases (held by the Swiss Institute of Bioinformatics (SIB) and the European Bioinformatics Institue (EBI) which are available at http://www.expasy.com/) and the GenBank database (held by the National Institute of Health (NIH) which is available at http://www.ncbi.nlm.nih.gov/GenBank/) provide protein sequences comprising the amino acid sequences listed for the VRPIs listed in Tables IV and V under the following accession numbers and each sequence is incorporated herein by reference. In many cases the protein sequences in the database will cross-reference a nucleic acid or gene sequence encoding the protein or related protein.

TABLE VI
Sequences encoding VRPIs or VRPI Related Proteins

| | | Rat/Mouse* | |
|---|---|---|---|
| | | Accession | Human Homologue |
| VRF | VRPI | Number | Accession Number |
| VRF-1 | VRPI-1 | P06399 | P02671 |
| VRF-14 | VRPI-14 | P06866 | P00739, P00738, P00737 |
| VRF-18 | VRPI-18 | P06866 | P00739, P00738, P00737 |
| VRF-19 | VRPI-19 | P02650 | P02649 |
| VRF-23 | VRPI-23 | P13635 | P00450 |
| VRF-28 | VRPI-28.1 | Q64291* | P35527 |
| VRF-28 | VRPI-28.2 | P05371 | P10909 |
| VRF-32 | VRPI-32.1 | P02535* | P13645 |
| VRF-32 | VRPI-32.2 | P20760 | — |
| VRF-36 | VRPI-36 | P02651 | P06727 |
| VRF-38 | VRPI-38 | P50446* | P13647 |
| VRF-43 | VRPI-43 | P02761 | — |
| VRF-47 | VRPI-47.1 | P02770 | P02768 |
| VRF-47 | VRPI-47.2 | P20059 | P02790 |
| VRF-58 | VRPI-58 | P02651 | P06727 |
| VRF-61 | VRPI-61 | P02770 | P02768 |
| VRF-63 | VRPI-63 | P02770 | P02768 |
| VRF-65 | VRPI-65 | P25030 | — |
| VRF-68 | VRPI-68.1 | P02650 | P02649 |
| VRF-68 | VRPI-68.2 | P05371 | P10909 |
| VRF-72 | VRPI-72 | P01026 | P01024 |
| VRF-73 | VRPI-73 | P26644 | P02749 |
| VRF-75 | VRPI-75 | P04276 | P02774 |
| VRF-86 | VRPI-86 | P20059 | P02790 |
| VRF-93 | VRPI-93 | P20059 | P02790 |
| VRF-94 | VRPI-94 | P97298* | P36955 |
| VRF-95 | VRPI-95 | P04639 | P02647 |
| VRF-100 | VRPI-100 | P14480 | P02675 |
| VRF-102 | VRPI-102 | — | P04217 |
| VRF-104 | VRPI-104 | P02651 | P06727 |
| VRF-106 | VRPI-106 | P04639 | P02647 |
| VRF-108 | VRPI-108 | P13020* | P06396 |
| VRF-115 | VRPI-115 | P02650 | P02649 |
| VRF-116 | VRPI-116 | P06399 | P02671 |
| VRF-118 | VRPI-118.1 | P01026 | P01024 |
| VRF-118 | VRPI-118.2 | P32261* | P01008 |
| VRF-120 | VRPI-120 | P20059 | P02790 |
| VRF-130 | VRPI-130 | P14480 | P02675 |
| VRF-183 | VRPI-183 | P18292 | P00734 |
| VRF-216 | VRPI-216 | P01026 | P01024 |
| VRF-245 | VRPI-245 | 11066005 | P04196 |
| VRF-249 | VRPI-249 | P12346 | P02787 |
| VRF-269 | VRPI-269 | P18292 | P00734 |

For any VRPI, degenerate probes, or probes taken from the sequences described above by accession number may be used for screening. In the case of degenerate probes, they can be constructed from the partial amino sequence information obtained from tandem mass spectra of tryptic digest peptides of the VRPI. To screen such a gene, any probe may be used that is complementary to the gene or its complement; preferably the probe is 10 nucleotides or longer, more preferably 15 nucleotides or longer. When a library is screened, clones with insert DNA encoding the VRPI or a fragment thereof will hybridize to one or more members of the corresponding set of degenerate oligonucleotide probes (or their complement). Hybridization of such oligonucleotide probes to genomic libraries is carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2X SSC, 1.0% SDS at 50° C. and washed using the washing conditions described supra for highly stringent or moderately stringent hybridization.

In yet another aspect of the invention, clones containing nucleotide sequences encoding the entire VRPI, a fragment of a VRPI, a VRPI-related polypeptide, or a fragment of a VRPI-related polypeptide any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed VRPI or VRPI-related polypeptides. In one embodiment, the various anti-VRPI antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes a VRPI, a fragment of a VRPI, a VRPI-related polypeptide, or a fragment of a VRPI-related polypeptide can be detected using DYNA Beads according to Olsvick et al., 29th ICAAC, Houston, Tex. 1989, incorporated herein by reference. Anti-VRPI antibodies are crosslinked to tosylated DYNA Beads M280, and these antibody-containing beads are then contacted with colonies or plaques expressing recombinant polypeptides. Colonies or plaques expressing a VRPI or VRPI-related polypeptide are identified as any of those that bind the beads.

Alternatively, the anti-VRPI antibodies can be nonspecifically immobilized to a suitable support, such as silica or Celite⁷ resin. This material is then used to adsorb to bacterial colonies expressing the VRPI protein or VRPI-related polypeptide as described herein.

In another aspect, PCR amplification may be used to isolate from genomic DNA a substantially pure DNA (i.e., a DNA substantially free of contaminating nucleic acids) encoding the entire VRPI or apart thereof Preferably such a DNA is at least 95% pure, more preferably at least 99% pure. Oligonucleotide sequences, degenerate or otherwise, that correspond to peptide sequences of VRPIs disclosed herein can be used as primers.

PCR can be carried out, e.g., by use of a Perkin-Elmer Cetus thermal cycler and Taq polymerase (Gene Am⁷ or AmpliTaq DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding a VRPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding a VRPI can also be identified by mRNA selection by nucleic acid hybridization followed by in vitro translation. In this procedure, fragments are used to isolate complementary mRNAs by hybridization. Such DNA fragments may represent available, purified DNA encoding a VRPI of another species (e~g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize a VRPI. A radiolabelled cDNA encoding a VRPI can be synthesized using the selected mRNA (from the adsorbed polysomes) as a template. The radiolabelled mRNA or cDNA may then be used as a probe to identify the DNA fragments encoding a VRPI from among other genomic DNA fragments.

Alternatives to isolating genomic DNA encoding a VRPI include, but are not limited to, chemically synthesizing the gene sequence itself from a known sequence or making cDNA to the mRNA which encodes the VRPI. For example, RNA for cDNA cloning of the gene encoding a VRPI can be isolated from cells which express the VRPI. Those skilled in the art will understand from the present description that other methods may be used and are within the scope of the invention.

Any suitable eukaryotic cell can serve as the nucleic acid source for the molecular cloning of the gene encoding a VRPI. The nucleic acid sequences encoding the VRPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U. K. Vol. I, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used.

As those skilled in the art will appreciate, the only limitation is that the vector system chosen be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding a VRPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the VRPI, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native VRPIs, nucleotide sequences encoding amino acid sequences with functionally equivalent amino acids, nucleotide sequences encoding VRPIs, a fragments of VRPIs, VRPI-related polypeptides, or fragments of VRPI-related polypeptides.

In a specific embodiment, an isolated nucleic acid molecule encoding a VRPI-related polypeptide can be created by introducing one or more nucleotide substitutions, additions or deletions into the nucleotide sequence of a VRPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

### 5.8 Expression of DNA Encoding VRPIs

The nucleotide sequence coding for a VRPI, a VRPI analog, a VRPI-related peptide, or a fragment or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the VRPI or its flanking regions, or the native gene encoding the VRPI-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a huma VRPI) is expressed. In yet another embodiment, a fragment of a VRPI comprising a domain of the VRPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding a VRPI or fragment thereof may be regulated by a second nucleic acid sequence so that the VRPI or fragment is expressed in a host transformed with the recombinant DNA molecule. For example, expression of a VRPI may be controlled by any promoter or enhancer element known in the art. Promoters which may be used to control the expression of the gene encoding a VRPI or a VRPI-related polypeptide include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the—lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Corn. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to a VRPI-encoding nucleic acid, one or more origins of replication, and, optionally, one or more selectable markers (e.g., an antibiotic resistance gene).

In a specific embodiment, an expression construct is made by subcloning a VRPI or a VRPI-related polypeptide coding sequence into the EcoRI restriction site of each of the three pGEX vectors (Glutathione S-Transferase expression vectors; Smith and Johnson, 1988, Gene 7:31-40). This allows for the expression of the VRPI product or VRPI-related polypeptide from the subclone in the correct reading frame.

In mammalian host cells, a number of viral-based expression systems may be utilized. In cases where an adenovirus is used as an expression vector, the VRPI coding sequence or VRPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a VRPI or a VRPI-related polypeptide can be identified by three general approaches: (a) nucleic acid hybridization, (b) presence or absence of “marker” gene functions, and (c) expression of inserted sequences. In the first approach, the presence of a gene encoding a VRPI inserted in an expression vector can be detected by nucleic acid hybridization using probes comprising sequences that are homologous to an inserted gene encoding a VRPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding a VRPI in the vector. For example, if the gene encoding the VRPI is inserted within the marker gene sequence of the vector, recombinants containing the gene encoding the VRPI insert can be identified by the absence of the marker gene function. In the third approach, recombinant expression vectors can be identified by assaying the gene product (i.e., VRPI) expressed by the recombinant. Such assays can be based, for example, on the physical or functional properties of the VRPI in in vitro assay systems, e.g., binding with anti-SPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered VRPI or VRPI-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, HEK293, 3T3, WI38, and in particular, endothelial cell lines, and normal human cell lines such as, for example, normal human endothelial cells. Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other specific embodiments, the VRPI, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding a VRPI, a fragment of a VRPI, a VRPI-related polypeptide, or a fragment of a VRPI-related polypeptide can fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

Fusion proteins can be made by ligating the appropriate nucleic acid sequences encoding the desired amino acid sequences to each other by methods known in the art, in the proper coding frame, and expressing the chimeric product by methods commonly known in the art. Alternatively, a fusion protein may be made by protein synthetic techniques, e.g., by use of a peptide synthesizer.

Both cDNA and genomic sequences can be cloned and expressed.

### 5.9 Domain Structure of VRPIs

Domains of some VRPIs are known in the art and have been described in the scientific literature. Moreover, domains of a VRPI can be identified using techniques known to those of skill in the art. For example, one or more domains of a VRPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED_form.html; Hofmann & Stoffel. (1993) ATMbase—A database of membrane spanning proteins segments.@ Biol. Chem. Hoppe-Seyler 347,166). The SAPS program analyzes polypeptides for statistically significant features like charge-clusters, repeats, hydrophobic regions, compositional domains (see, e.g., Brendel et al., 1992, Proc. Natl. Acad. Sci. USA 89: 2002-2006). Thus, based on the present description, the skilled artisan can identify domains of a VRPI having enzymatic or binding activity, and further can identify nucleotide sequences encoding such domains. These nucleotide sequences can then be used for recombinant expression of a VRPI fragment that retains the enzymatic or binding activity of the VRPI.

Based on the present description, the skilled artisan can identify domains of a VRPI having enzymatic or binding activity, and further can identify nucleotide sequences encoding such domains. These nucleotide sequences can then be used for recombinant expression of VRPI fragments that retain the enzymatic or binding activity of the VRPI.

In one embodiment, a VRPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

A VRPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay. In a preferred embodiment, the function of a domain of a VRPI is determined using an assay described in one or more of the references identified in Table VIII infra.

### 5.10 Production of Antibodies to VRPIs

According to the invention a VRPI, VRPI analog, VRPI-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab=) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, antibodies that recognize gene products of genes encoding VRPIs are publicly available. For example, antibodies that recognize these VRPIs and/or their isoforms include antibodies recognizing VRPI-1, VRPI-19, VRPI-23, VRPI-28.1, VRPI-28.2, VRPI-32.1, VRPI-36, VRPI-38, VRPI-47.1, VRPI-47.2, VRPI-58, VRPI-61, VRPI-63, VRPI-68.1, VRPI-68.2, VRPI-72, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-269, which antibodies may be purchased from commercial sources as described above. In another embodiment, methods known to those skilled in the art are used to produce antibodies that recognize a VRPI, a VRPI analog, a VRPI-related polypeptide, or a derivative or fragment of any of the foregoing.

In one embodiment of the invention, antibodies to a specific domain of a VRPI are produced. In a specific embodiment, hydrophilic fragments of a VRPI are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of a VRPI, one may assay generated hybridomas for a product which binds to a VRPI fragment containing such domain. For selection of an antibody that specifically binds a first VRPI homolog but which does not specifically bind to (or binds less avidly to) a second VRPI homolog, one can select on the basis of positive binding to the first VRPI homolog and a lack of binding to (or reduced binding to) the second VRPI homolog. Similarly, for selection of an antibody that specifically binds a VRPI but which does not specifically bind to (or binds less avidly to) a different isoform of the same protein (such as a different glycoform having the same core peptide as the VRPI), one can select on the basis of positive binding to the VRPI and a lack of binding to (or reduced binding to) the different isoform (e.g., a different glycoform). Thus, the present invention provides an antibody (preferably a monoclonal antibody) that binds with greater affinity (preferably at least 2-fold, more preferably at least 5-fold still more preferably at least 10-fold greater affinity) to a VRPI than to a different isoform or isoforms (e.g., glycoforms) of the VRPI.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to a VRPI, a fragment of a VRPI, a VRPI-related polypeptide, or a fragment of a VRPI-related polypeptide. In a particular embodiment, rabbit polyclonal antibodies to an epitope of a VRPI or a VRPI-related polypeptide can be obtained. For example, for the production of polyclonal or monoclonal antibodies, various host animals can be immunized by injection with the native or a synthetic (e.g. recombinant) version of a VRPI, a fragment of a VRPI, a VRPI-related polypeptide, or a fragment of a VRPI-related polypeptide, including but not limited to rabbits, mice, rats, etc. The Preferred Technology described herein provides isolated VRPIs suitable for such immunization. If the VRPI is purified by gel electrophoresis, the VRPI can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward a VRPI, a fragment of a VRPI, a VRPI-related polypeptide, or a fragment of a VRPI-related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). A chimeric antibody is a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a VRPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labelled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al., Methods in Enzymology,1986, 121:210.

The invention provides functionally active fragments, derivatives or analogs of the anti-VRPI immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)₂ fragments and Fab fragments. Antibody fragments which recognize specific epitopes may be generated by known techniques. F(ab′)₂ fragments consist of the variable region, the light chain constant region and the CH1 domain of the heavy chain and are generated by pepsin digestion of the antibody molecule. Fab fragments are generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of the VRPIs of the invention, e.g., for imaging these proteins, measuring levels thereof in appropriate physiological samples, in diagnostic methods, etc.

### 5.11 Expression Of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, NY) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 198, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, HEK293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+nitriloacetic acid-agarose columns and histidine-tagged proteins are selectively eluted with imidazole-containing buffers.

### 5.12 Conjugated Antibodies

In a preferred embodiment, anti-VRPI antibodies or fragments thereof are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

An anti-VRPI antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-blood), granulocyte colony stimulating factor (G-blood), nerve growth factor (NGF) or other growth factor.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

### 5.13 Diagnosis of Vascular Response

In accordance with the present invention, test samples of blood, serum, plasma or urine obtained from a subject suspected of having or known to have vascular response can be used for diagnosis or monitoring. In one embodiment, a decreased abundance of one or more VRFs or VRPIs (or any combination of them) in a test sample relative to a control sample (from a subject or subjects free from vascular response) or a previously determined reference range indicates the presence of vascular response; VRFs and VRPIs suitable for this purpose are identified in Tables I and IV, respectively, as described in detail above. In another embodiment of the invention, an increased abundance of one or more VRFs or VRPIs (or any combination of them) in a test sample compared to a control sample or a previously determined reference range indicates the presence of vascular response; VRFs and VRPIs suitable for this purpose are identified in Tables II and V, respectively, as described in detail above. In another embodiment, the relative abundance of one or more VRFs or VRPIs (or any combination of them) in a test sample compared to a control sample or a previously determined reference range indicates a subtype of vascular response (e.g., familial or sporadic vascular response). In yet another embodiment, the relative abundance of one or more VRFs or VRPIs (or any combination of them) in a test sample relative to a control sample or a previously determined reference range indicates the degree or severity of vascular response. In any of the aforesaid methods, detection of one or more VRPIs described herein may optionally be combined with detection of one or more additional biomarkers for vascular response including, but not limited to apolipoprotein A1 (Apo E). Any suitable method in the art can be employed to measure the level of VRFs and VRPIs, including but not limited to the Preferred Technology described herein, kinase assays, immunoassays to detect and/or visualize the VRPI (e.g., Western blot, immunoprecipitation followed by sodium dodecyl sulfate polyacrylamide gel electrophoresis, immunocytochemistry, etc.). In cases where a VRPI has a known function, an assay for that function may be used to measure VRPI expression. In a further embodiment, a decreased abundance of mRNA including one or more VRPIs identified in Table IV (or any combination of them) in a test sample relative to a control sample or a previously determined reference range indicates the presence of vascular response. In yet a further embodiment, an increased abundance of mRNA encoding one or more VRPIs identified in Table V (or any combination of them) in a test sample relative to a control sample or previously determined reference range indicates the presence of vascular response. Any suitable hybridization assay can be used to detect VRPI expression by detecting and/or visualizing mRNA encoding the VRPI (e.g., Northern assays, dot blots, in situ hybridization, etc.).

In another embodiment of the invention, labeled antibodies, derivatives and analogs thereof, which specifically bind to a VRPI can be used for diagnostic purposes to detect, diagnose, or monitor vascular response. Preferably, vascular response is detected in an animal, more preferably in a mammal and most preferably in a human.

### 5.14 Screening Assays

The invention provides methods for identifying agents (e.g. drug candidiates or test compounds) or environmental factors that bind to a VRPI or have a stimulatory or inhibitory effect on the expression or activity of a VRPI. The invention also provides methods of identifying agents that bind to a VRPI-related polypeptide or a VRPI fusion protein or have a stimulatory or inhibitory effect on the expression or activity of a VRPI-related polypeptide or a VRPI fusion protein.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No.5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment agents that do or do not interact with (i.e., bind to) a VRPI, a VRPI fragment (e.g. a functionally active fragment), a VRPI-related polypeptide, a fragment of a VRPI-related polypeptide, or a VRPI fusion protein are identified in a cell-based assay system. In accordance with this embodiment, cells expressing a VRPI, a fragment of a VRPI, a VRPI-related polypeptide, a fragment of a VRPI-related polypeptide, or a VRPI fusion protein are contacted with an agent, such as a drug candidate, or a control and the ability of the agent to interact with the VRPI is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the VRPI, fragment of the VRPI, VRPI-related polypeptide, a fragment of the VRPI-related polypeptide, or a VRPI fusion protein endogenously or be genetically engineered to express the VRPI, fragment of the VRPI, VRPI-related polypeptide, a fragment of the VRPI-related polypeptide, or a VRPI fusion protein. In certain instances, the VRPI, fragment of the VRPI, VRPI-related polypeptide, a fragment of the VRPI-related polypeptide, or a VRPI fusion protein or the candidate compound is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a VRPI and an agent, such as a drug candidate. The ability of the candidate compound to interact directly or indirectly with a VRPI, a fragment of a VRPI, a VRPI-related polypeptide, a fragment of a VRPI-related polypeptide, or a VRPI fusion protein can be determined by methods known to those of skill in the art. For example, the interaction between an agentand a VRPI, a fragment of a VRPI, a VRPI-related polypeptide, a fragment of a VRPI-related polypeptide, or a VRPI fusion protein can be determined by flow cytometry, a scintillation assay, immunoprecipitation or western blot analysis.

In another embodiment, agents that do or do not interact with (i.e., bind to) a VRPI, a VRPI fragment (e.g., a functionally active fragment) a VRPI-related polypeptide, a fragment of a VRPI-related polypeptide, or a VRPI fusion protein are identified in a cell-free assay system. In accordance with this embodiment, a native or recombinant VRPI or fragment thereof, or a native or recombinant VRPI-related polypeptide or fragment thereof, or a VRPI-fusion protein or fragment thereof, is contacted with an agent or a control and the ability of the agent to interact with the VRPI or VRPI-related polypeptide, or VRPI fusion protein is determined. If desired, this assay may be used to screen a plurality (e.g. a library) of agents. Preferably, the VRPI, VRPI fragment, VRPI-related polypeptide, a fragment of a VRPI-related polypeptide, or a VRPI-fusion protein is first immobilized, by, for example, contacting the VRPI, VRPI fragment, VRPI-related polypeptide, a fragment of a VRPI-related polypeptide, or a VRPI fusion protein with an immobilized antibody which specifically recognizes and binds it, or by contacting a purified preparation of the VRPI, VRPI fragment, VRPI-related polypeptide, fragment of a VRPI-related polypeptide, or a VRPI fusion protein with a surface designed to bind proteins. The VRPI, VRPI fragment, VRPI-related polypeptide, a fragment of a VRPI-related polypeptide, or a VRPI fusion protein may be partially or completely purified (e.g., partially or completely free of other polypeptides) or part of a cell lysate. Further, the VRPI, VRPI fragment, VRPI-related polypeptide, a fragment of a VRPI-related polypeptide may be a fusion protein comprising the VRPI or a biologically active portion thereof, or VRPI-related polypeptide and a domain such as glutathionine-S-transferase. Alternatively, the VRPI, VRPI fragment, VRPI-related polypeptide, fragment of a VRPI-related polypeptide or VRPI fusion protein can be biotinylated using techniques well known to those of skill in the art (e.g., biotinylation kit, Pierce Chemicals; Rockford, Ill.). The ability of the agent to interact with a VRPI, VRPI fragment, VRPI-related polypeptide, a fragment of a VRPI-related polypeptide, or a VRPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of a VRPI or is responsible for the post-translational modification of a VRPI. In a primary screen, a plurality (e.g., a library) of agents e.g., drug candidates, are contacted with cells that naturally or recombinantly express: (i) a VRPI, an isoform of a VRPI, a VRPI homolog a VRPI-related polypeptide, a VRPI fusion protein, or a biologically active fragment of any of the foregoing; and (ii) a protein that is responsible for processing of the VRPI, VRPI isoform, VRPI homolog, VRPI-related polyp eptide, VRPI fusion protein, or fragment in order to identify compounds that modulate the production, degradation, or post-translational modification of the VRPI, VRPI isoform, VRPI homolog, VRPI-related polypeptide, VRPI fusion protein or fragment. If desired, agents identified in the primary screen can then be assayed in a secondary screen against cells naturally or recombinantly expressing the specific VRPI of interest. The ability of the agent to modulate the production, degradation or post-translational modification of a VRPI, isoform, homolog, VRPI-related polypeptide, or VRPI fusion protein can be determined by methods known to those of skill in the art, including without limitation, flow cytometry, a scintillation assay, immunoprecipitation and western blot analysis.

In another embodiment, agents that do or do not competitively interact with (i.e., bind to) a VRPI, VRPI fragment, VRPI-related polypeptide, a fragment of a VRPI-related polypeptide, or a VRPI fusion protein are identified in a competitive binding assay. In accordance with this embodiment, cells expressing a VRPI, VRPI fragment, VRPI-related polypeptide, a fragment of a VRPI-related polypeptide, or a VRPI fusion protein are contacted with an agents and a compound known to interact with the VRPI, VRPI fragment, VRPI-related polypeptide, a fragment of a VRPI-related polypeptide or a VRPI fusion protein; the ability of the agent to competitively interact with the VRPI, VRPI fragment, VRPI-related polypeptide, fragment of a VRPI-related polypeptide, or a VRPI fusion protein is then determined. Alternatively, agents that competitively interact with (i.e., bind to) a VRPI, VRPI fragment, VRPI-related polypeptide or fragment of a VRPI-related polypeptide are identified in a cell-free assay system by contacting a VRPI, VRPI fragment, VRPI-related polypeptide, fragment of a VRPI-related polypeptide, or a VRPI fusion protein with a candidate agent and a compound known to interact with the VRPI, VRPI-related polypeptide or VRPI fusion protein. As stated above, the ability of the candidate agent to interact with a VRPI, VRPI fragment, VRPI-related polypeptide, a fragment of a VRPI-related polypeptide, or a VRPI fusion protein can be determined by methods known to those of skill in the art. These assays, whether cell-based or cell-free, can be used to screen a plurality (e.g., a library) of candidate agents.

In another embodiment, agents that do or do not modulate (i.e., upregulate or downregulate) the expression of a VRPI, or a VRPI-related polypeptide are identified by contacting cells (e.g., cells of prokaryotic origin or eukaryotic origin) expressing the VRPI, or VRPI-related polypeptide with a candidate agent or a control (e.g., phosphate buffered saline (PBS)) and determining the expression of the VRPI, VRPI-related polypeptide, or VRPI fusion protein, mRNA encoding the VRPI, or MRNA encoding the VRPI-related polypeptide. The level of expression of a selected VRPI, VRPI-related polypeptide, mRNA encoding the VRPI, or MRNA encoding the VRPI-related polypeptide in the presence of the candidate agent is compared to the level of expression of the VRPI, VRPI-related polypeptide, mRNA encoding the VRPI, or mRNA encoding the VRPI-related polypeptide in the absence of the candidate agent (e.g., in the presence of a control). The candidate agent can then be identified as a modulator of the expression of the VRPI, or a VRPI-related polypeptide based on this comparison. For example, when expression of the VRPI or MRNA is significantly greater in the presence of the candidate agent than in its absence, the candidate agent is identified as a stimulator of expression of the VRPI or mRNA. Alternatively, when expression of the VRPI or MRNA is significantly less in the presence of the candidate agent than in its absence, the candidate agent is identified as an inhibitor of the expression of the VRPI or mRNA. The level of expression of a VRPI or the mRNA that encodes it can be determined by methods known to those of skill in the art. For example, mRNA expression can be assessed by Northern blot analysis or RT-PCR, and protein levels can be assessed by western blot analysis.

In another embodiment, agents that do or do not modulate the activity of a VRPI, or a VRPI-related polypeptide are identified by contacting a preparation containing the VRPI or VRPI-related polypeptide, or cells (e.g., prokaryotic or eukaryotic cells) expressing the VRPI or VRPI-related polypeptide with a test agent or a control and determining the ability of the test agent to modulate (e.g., stimulate or inhibit) the activity of the VRPI or VRPI-related polypeptide. The activity of a VRPI or a VRPI-related polypeptide can be assessed by detecting induction of a cellular signal transduction pathway of the VRPI or VRPI-related polypeptide (e.g. , intracellular Ca2+, diacylglycerol, IP3, etc.), detecting catalytic or enzymatic activity of the target on a suitable substrate, detecting the induction of a reporter gene (e.g., a regulatory element that is responsive to a VRPI or a VRPI-related polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639, which is incorporated herein by reference). The candidate agent can then be identified as a modulator of the activity of a VRPI or VRPI-related polypeptide by comparing the effects of the candidate agent to the control. Suitable control compounds include phosphate buffered saline (PBS) and normal saline (NS).

In another embodiment, agents that do or do not modulate (i.e., upregulate or downregulate) the expression, activity or both the expression and activity of a VRPI or VRPI-related polypeptide are identified in an animal model. Examples of suitable animals include, but are not limited to, mice, rats, rabbits, monkeys, guinea pigs, dogs and cats. Preferably, the animal used represent a model of vascular response. In accordance with this embodiment, the test agent or a control is administered (e.g., orally, rectally or parenterally such as intraperitoneally or intravenously) to a suitable animal and the effect on the expression, activity or both expression and activity of the VRPI or VRPI-related polypeptide is determined. Changes in the expression of a VRPI or VRPI-related polypeptide can be assessed by the methods outlined above. As the method for screening drug candidates for their potential to induce a vascular response, the agents tested are advantageously agents which will be administered systemically, e.g., intravenously, since it is such agents that are most likely to induce an unwanted vascular response e.g., vasculitis.

In yet another embodiment, a VRPI or VRPI-related polypeptide is used as a “bait protein” in a two-hybrid assay or three hybrid assay to identify other proteins that bind to or interact with a VRPI or VRPI-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the VRPIs of the invention as, for example, upstream or downstream elements of a signaling pathway involving the VRPIs of the invention.

Table VIII enumerates scientific publications describing suitable assays for detecting or quantifying enzymatic or binding activity of a VRPI, a VRPI analog, a VRPI-related polypeptide, or a fragment of any of the foregoing. Each such reference is hereby incorporated in its entirety. In a preferred embodiment, as assay referenced in Table VII is used in the screens and assays described herein, for example to screen for or identify a compound that modulates the activity of (or that modulates both the expression and activity of) a VRPI, VRPI analog, or VRPI-related polypeptide, a fragment of any of the foregoing.

TABLE VIII
Scientific Publications for Assays

| VRF | VRPI | Scientific References |
|---|---|---|
| VRF-72 | VRPI-72 | “Time-resolved immunofluorometric assay of |
| | | complement C3: application to cerebrospinal |
| | | fluid.” |
| VRF-118 | VRPI-118.1 | Clin Chem 1993 Feb 39:2 309-12 |
| VRF-216 | VRPI-216 | “A fluorimetric assay for native C3. The |
| | | hemolytically active form of the third com- |
| | | ponent of human complement.” |

More particularly, in one aspect, the invention provides methods for the identification of agents which will not have an effect on the expression or activity of a VRPI, VRPI-related polypeptide or VRPI fusion protein, and as such will not induce a vascular response. When such agents are drug candidiates they can be propgressed into development with a greater level of cvonfidence that they will not produce unwanted vascular responses when administered clinically.

This aspect of the invention allows for toxicity screening to be carried out at a much earlier stage. In particular, it can show whether agent will or will not induce vascular response. In relation to the screening of agents for their potential to induce an unwanted vascular response, The term “agent” is used herein to describe a wide variety of physical, chemical or biological factors. For example, physical agents include, without limitation, the diet of a subject, a change in temperature or humidity, exposure to ultraviolet radiation and the like. Biological and chemical agents include exogenous factors such as pharmaceutical compounds (including candidate compounds and test compounds), toxic compounds, proteins, peptides, chemical compositions, natural pathogens, such as microbial agents including bacteria, viruses and lower eukaryotic cells such as fungi, yeast and simple multicellular organisms, as well as endogenous factors which occur naturally in the body, including, without limitation, hormones, enzymes, receptors, ligands and the like, which may or may not be recombinant. This invention further provides novel agents identified by the above-described screening assays and uses thereof for treatments as described herein.

### 5.15 Therapeutic Uses of VRPIs

The invention provides for treatment or prevention of various diseases and disorders by administration of a therapeutic compound. Such compounds include but are not limited to: VRPIs, VRPI analogs, VRPI-related polypeptides and derivatives (including fragments) thereof; antibodies to the foregoing; nucleic acids encoding VRPIs, VRPI analogs, VRPI-related polypeptides and fragments thereof; antisense nucleic acids to a gene encoding a VRPI or VRPI-related polypeptide; and modulator (e.g., agonists and antagonists) of a gene encoding a VRPI or VRPI-related polypeptide. An important feature of the present invention is the identification of genes encoding VRPIs involved in vascular response. Vascular response can be treated (e.g. to ameliorate symptoms or to retard onset or progression) or prevented by administration of a therapeutic compound that promotes function or expression of one or more VRPIs that are decreased in the blood of vascular response subjects having vascular response, or by administration of a therapeutic compound that reduces function or expression of one or more VRPIs that are increased in the blood of subjects having vascular response.

In one embodiment, one or more antibodies each specifically binding to a VRPI are administered alone or in combination with one or more additional therapeutic compounds or treatments.

Preferably, a biological product such as an antibody is allogeneic to the subject to which it is administered. In a preferred embodiment, a human VRPI or a human VRPI-related polypeptide, a nucleotide sequence encoding a human VRPI or a human VRPI-related polypeptide, or an antibody to a human VRPI or a human VRPI-related polypeptide, is administered to a human subject for therapy (e.g. to ameliorate symptoms or to retard onset or progression) or prophylaxis.

### 5.15.1 Treatment And Prevention Of Vascular Response

Vascular response is treated or prevented by administration to a subject suspected of having or known to have vascular response or to be at risk of developing vascular response of a compound that modulates (i.e., increases or decreases) the level or activity (i.e., function) of one or more VRPIs—or the level of one or more VRFs—that are differentially present in the blood of subjects having vascular response compared with blood of subjects free from vascular response. In one embodiment, vascular response is treated or prevented by administering to a subject suspected of having or known to have vascular response or to be at risk of developing vascular response a compound that upregulates (i.e., increases) the level or activity (i.e., function) of one or more VRPIs—or the level of one or more VRFs—that are decreased in the blood of subjects having vascular response. In another embodiment, a compound is administered that upregulates the level or activity (i.e., function) of one or more VRPIs—or the level of one or more VRFs—that are increased in the blood of subjects having vascular response. Examples of such a compound include but are not limited to: VRPIs, VRPI fragments and VRPI-related polypeptides; nucleic acids encoding a VRPI, a VRPI fragment and a VRPI-related polypeptide (e.g., for use in gene therapy); and, for those VRPIs or VRPI-related polypeptides with enzymatic activity, compounds or molecules known to modulate that enzymatic activity. Other compounds that can be used, e.g., VRPI agonists, can be identified using in vitro assays.

Vascular response is also treated or prevented by administration to a subject suspected of having or known to have vascular response or to be at risk of developing vascular response of a compound that downregulates the level or activity of one or more VRPIs —or the level of one or more VRFs—that are increased in the blood of subjects having vascular response. In another embodiment, a compound is administered that downregulates the level or activity of one or more VRPIs—or the level of one or more VRFs—that are decreased in the blood of subjects having vascular response. Examples of such a compound include, but are not limited to, VRPI antisense oligonucleotides, ribozymes, antibodies directed against VRPIs, and compounds that inhibit the enzymatic activity of a VRPI. Other useful compounds e.g., VRPI antagonists and small molecule VRPI antagonists, can be identified using in vitro assays.

In a preferred embodiment, therapy or prophylaxis is tailored to the needs of an individual subject. Thus, in specific embodiments, compounds that promote the level or function of one or more VRPIs, or the level of one or more VRFs, are therapeutically or prophylactically administered to a subject suspected of having or known to have vascular response, in whom the levels or functions of said one or more VRPIs, or levels of said one or more VRFs, are absent or are decreased relative to a control or normal reference range. In further embodiments, compounds that promote the level or function of one or more VRPIs, or the level of one or more VRFs, are therapeutically or prophylactically administered to a subject suspected of having or known to have vascular response in whom the levels or functions of said one or more VRPIs, or levels of said one or more VRFs, are increased relative to a control or to a reference range. In further embodiments, compounds that decrease the level or function of one or more VRPIs, or the level of one or more VRFs, are therapeutically or prophylactically administered to a subject suspected of having or known to have vascular response in whom the levels or functions of said one or more VRPIs, or levels of said one or more VRFs, are increased relative to a control or to a reference range. In further embodiments, compounds that decrease the level or function of one or more VRPIs, or the level of one or more VRFs, are therapeutically or prophylactically administered to a subject suspected of having or known to have vascular response in whom the levels or functions of said one or more VRPIs, or levels of said one or more VRFs, are decreased relative to a control or to a reference range. The change in VRPI function or level, or VRF level, due to the administration of such compounds can be readily detected, e.g., by obtaining a sample (e.g., a sample of blood, blood or urine or a tissue sample such as biopsy tissue) and assaying in vitro the levels of said VRFs or the levels or activities of said VRPIs, or the levels of mRNAs encoding said VRPIs. or any combination of the foregoing. Such assays can be performed before and after the administration of the compound as described herein.

The compounds of the invention include but are not limited to any compound, e.g., a small organic molecule, protein, peptide, antibody, nucleic acid, etc. that restores the vascular response VRPI or VRF profile towards normal.

### 5.15.2 Gene Therapy

In a specific embodiment, nucleic acids comprising a sequence encoding a VRPI, a VRPI fragment, VRPI-related polypeptide or fragment of a VRPI-related polypeptide, are administered to promote VRPI function by way of gene therapy. Gene therapy refers to administration to a subject of an expressed or expressible nucleic acid. In this embodiment, the nucleic acid produces its encoded polypeptide that mediates a therapeutic effect by promoting VRPI function.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11 (5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the compound comprises a nucleic acid encoding a VRPI or fragment or chimeric protein thereof, said nucleic acid being part of an expression vector that expresses a VRPI or fragment or chimeric protein thereof in a suitable host. In particular, such a nucleic acid has a promoter operably linked to the VRPI coding region, said promoter being inducible or constitutive (and, optionally, tissue-specific). In another particular embodiment, a nucleic acid molecule is used in which the VRPI coding sequences and any other desired sequences are flanked by regions that promote homologous recombination at a desired site in the genome, thus providing for intrachromosomal expression of the VRPI nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject; this approach is known as ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains a nucleic acid encoding a VRPI is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the VRPI to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding a VRPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Direct injection of a DNA coding for a VRPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding a VRPI and (b) a promoter are injected into a subject to elicit an immune response to the VRPI.

### 5.15.3 Inhibition of VRPIs to Treat Vascular Response

In one embodiment of the invention, vascular response is treated or prevented by administration of a compound that antagonizes (inhibits) the level(s) and/or function(s) of one or more VRPIs which are elevated in the blood of subjects having vascular response as compared with blood of subjects free from vascular response. Compounds useful for this purpose include but are not limited to anti-VRPI antibodies (and fragments and derivatives containing the binding region thereof), VRPI antisense or ribozyme nucleic acids, and nucleic acids encoding dysfunctional VRPIs that are used to “knockout” endogenous VRPI function by homologous recombination (see, e.g., Capecchi, 1989, Science 244:1288-1292). Other compounds that inhibit VRPI function can be identified by use of known in vitro assays, e.g., assays for the ability of a test compound to inhibit binding of a VRPI to another protein or a binding partner, or to inhibit a known VRPI function. Preferably such inhibition is assayed in vitro or in cell culture, but genetic assays may also be employed. The Preferred Technology can also be used to detect levels of the VRPI before and after the administration of the compound. Preferably, suitable in vitro or in vivo assays are utilized to determine the effect of a specific compound and whether its administration is indicated for treatment of the affected tissue, as described in more detail below.

In a specific embodiment, a compound that inhibits a VRPI function is administered therapeutically or prophylactically to a subject in whom an increased blood level or functional activity of the VRPI (e.g., greater than the normal level or desired level) is detected as compared with blood of subjects free from vascular response or a predetermined reference range. Methods standard in the art can be employed to measure the increase in a VRPI level or function, as outlined above. Preferred VRPI inhibitor compositions include small molecules, i.e., molecules of 1000 daltons or less. Such small molecules can be identified by the screening methods described herein.

### 5.15.4 Antisense Regulation of VRPIs

In a specific embodiment, VRPI expression is inhibited by use of VRPI antisense nucleic acids. The present invention provides the therapeutic or prophylactic use of nucleic acids comprising at least six nucleotides that are antisense to a gene or cDNA encoding a VRPI or a portion thereof. As used herein, a VRPI “antisense” nucleic acid refers to a nucleic acid capable of hybridizing by virtue of some sequence complementarity to a portion of an RNA (preferably mRNA) encoding a VRPI. The antisense nucleic acid may be complementary to a coding and/or noncoding region of an mRNA encoding a VRPI. Such antisense nucleic acids have utility as compounds that inhibit VRPI expression, and can be used in the treatment or prevention of vascular response.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising an effective amount of the VRPI antisense nucleic acids of the invention in a pharmaceutically acceptable carrier, as described infra.

In another embodiment, the invention provides methods for inhibiting the expression of a VRPI nucleic acid sequence in a prokaryotic or eukaryotic cell comprising providing the cell with an effective amount of a composition comprising a VRPI antisense nucleic acid of the invention.

VRPI antisense nucleic acids and their uses are described in detail below.

### 5.15.5 VRPI Antisense Nucleic Acids

The VRPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, a VRPI antisense oligonucleotide is provided, preferably of single-stranded DNA. The oligonucleotide may be modified at any position on its structure with substituents generally known in the art.

The VRPI antisense oligonucleotide may comprise at least one of the following modified base moieties: 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyarninomethyl-2-thiouracil, beta-D-mannosylqueosine, 5N-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an -anomeric oligonucleotide. An -anomeric oligonucleotide forms specific double-stranded hybrids with complementary RNA in which, contrary to the usual -units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In a specific embodiment, the VRPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the

1. A method for screening, diagnosis or prognosis of Vascular Response in a subject, for determining the stage or severity of Vascular Response in a subject, for identifying a subject at risk of developing Vascular Response, or for monitoring the effect of therapy administered to a subject having Vascular Response, said method comprising:
(a) analyzing a test sample of a body fluid or tissue from the subject by two dimensional electrophoresis to generate a two-dimensional array of features, said array comprising at least one chosen feature whose relative abundance correlates with the presence, absence, stage or severity of Vascular Response or predicts the onset or course of Vascular Response; and
(b) comparing the abundance of each chosen feature in the test sample with the abundance of that chosen feature in body fluid from one or more subjects free from Vascular Response, or with a previously determined reference range for that feature in subjects free from Vascular Response, or with the abundance at least one Expression Reference Feature (ERF) in the test sample.

2. The method of claim 1, wherein the body fluid is blood.

3. The method of claim 1 or claim 2, wherein said method is for screening or diagnosis of Vascular Response and the relative abundance of at least one chosen feature correlates with the presence or absence of Vascular Response.

4. The method of claim 1 or claim 2, wherein said method is for monitoring the effect of therapy administered to a subject having Vascular Response and the relative abundance of at least one chosen feature correlates with the severity of Vascular Response.

5. The method of claim 1 or claim 2, wherein step (b) comprises comparing the abundance of each chosen feature in the sample with the abundance of that chosen feature in a sample from one or more subjects free from Vascular Response or with a previously determined reference range for that chosen feature in subjects free from Vascular Response.

6. The method according to claim 1 or claim 2, wherein step (b) comprises quantitatively detecting one or more of the following Vascular Response-Associated Features (VRFs): VRF-1, VRF-2, VRF-3, VRF-4, VRF-5, VRF-6, VRF-7, VRF-8, VRF-9, VRF-10, VRF-11, VRF-12, VRF-13, VRF-14, VRF-15, VRF-16, VRF-17, VRF-18, VRF-19, VRF-20, VRF-21, VRF-22, VRF-23, VRF-24, VRF-25, VRF-26, VRF-27, VRF-28, VRF-29, VRF-30, VRF-31, VRF-32, VRF-34, VRF-35, VRF-36, VRF-37, VRF-38, VRF-40, VRF-42, VRF-43, VRF-44, VRF-45, VRF-46, VRF-47, VRF-48, VRF-50, VRF-51, VRF-52, VRF-53, VRF-54, VRF-55, VRF-56, VRF-57, VRF-58, VRF-59, VRF-60, VRF-61, VRF-62, VRF-63, VRF-64, VRF-65, VRF-66, VRF-67, VRF-68, VRF-69, VRF-70, VRF-71, VRF-72, VRF-73, VRF-74, VRF-75, VRF-76, VRF-77, VRF-79, VRF-80, VRF-81, VRF-82, VRF-83, VRF-84, VRF-85, VRF-86, VRF-87, VRF-88, VRF-89, VRF-90, VRF-91, VRF-92, VRF-93, VRF-94, VRF-95, VRF-96, VRF-97, VRF-98, VRF-99, VRF-100, VRF-101, VRF-102, VRF-103, VRF-104, VRF-105, VRF-106, VRF-107, VRF-108, VRF-109, VRF-110, VRF-111, VRF-112, VRF-113, VRF-114, VRF-115, VRF-116, VRF-117, VRF-118, VRF-119, VRF-120, VRF-122, VRF-123, VRF-124, VRF-125, VRF-126, VRF-127, VRF-128, VRF-129, VRF-130, VRF-131, VRF-132, VRF-133, VRF-134, VRF-135, VRF-136, VRF-137, VRF-138, VRF-139, VRF-140, VRF-141, VRF-142, VRF-143, VRF-144, VRF-145, VRF-146, VRF-147, VRF-148, VRF-149, VRF-150, VRF-151, VRF-152, VRF-153, VRF-154, VRF-155, VRF-156, VRF-157, VRF-158, VRF-159, VRF-160, VRF-161, VRF-162, VRF-163, VRF-164, VRF-165, VRF-166, VRF-167, VRF-168, VRF-169, VRF-170, VRF-171, VRF-172, VRF-173, VRF-174, VRF-175, VRF-176, VRF-177, VRF-178, VRF-179, VRF-180, VRF-181, VRF-182, VRF-183, VRF-184, VRF-185, VRF-186, VRF-187, VRF-188, VRF-189, VRF-190, VRF-191, VRF-192, VRF-193, VRF-194, VRF-195, VRF-196, VRF-197, VRF-198, VRF-199, VRF-200, VRF-201, VRF-202, VRF-203, VRF-204, VRF-205, VRF-206, VRF-207, VRF-208, VRF-209, VRF-210, VRF-211, VRF-212, VRF-213, VRF-214, VRF-215, VRF-216, VRF-217, VRF-218, VRF-219, VRF-220, VRF-221, VRF-222, VRF-223, VRF-224, VRF-225, VRF-226, VRF-227, VRF-228, VRF-229, VRF-230, VRF-231, VRF-232, VRF-233, VRF-234, VRF-235, VRF-236, VRF-237, VRF-238, VRF-239, VRF-240, VRF-241, VRF-242, VRF-243, VRF-244, VRF-245, VRF-246, VRF-247, VRF-248, VRF-249, VRF-250, VRF-251, VRF-252, VRF-253, VRF-254, VRF-255, VRF-256, VRF-257, VRF-258, VRF-259, VRF-260, VRF-261, VRF-262, VRF-263, VRF-264, VRF-265, VRF-266, VRF-267, VRF-268, VRF-269, VRF-270, VRF-271, VRF-272,

7. The method according to claim 1 or claim 2, wherein step (a) comprises isoelectric focussing followed by sodium dodecyl sulfate polyacrylamide gel electrophoresis (SDS-PAGE).

8. A method for screening, diagnosis or prognosis of Vascular Response in a subject, for determining the stage or severity of Vascular Response in a subject, for identifying a subject at risk of developing Vascular Response, or for monitoring the effect of therapy administered to a subject having Vascular Response, said method comprising quantitatively detecting, in a sample from the subject, at least one of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269,

9. The method according to claim 8, wherein the step of quantitatively detecting comprises testing at least one aliquot of the sample, said step of testing comprising:
(a) contacting the aliquot with an antibody that is immunospecific for a preselected VRPI; and
(b) quantitatively measuring any binding that has occurred between the antibody and at least one species in the aliquot.

10. The method according to claim 9, wherein the antibody is a monoclonal antibody.

11. The method according to claim 9, wherein the step of quantitatively detecting comprises testing a plurality of aliquots with a plurality of antibodies for quantitative detection of a plurality of preselected VRPIs.

12. The method according to claim 11, wherein the antibodies are monoclonal antibodies.

13. A preparation comprising one or more of the following recombinant or isolated Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269,

14. A preparation of claim 13, wherein the one or more VRPIs are in a recombinant form.

15. A kit comprising the preparation of claim 13 or 14.

16. A kit comprising a plurality VRPIs, wherein each VRPI is a VRPI of claim 13 or claim 14.

17. An antibody capable of immunospecific binding to one of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269 or an ortholog thereof.

18. The antibody of claim 17, which is a monoclonal antibody.

19. The antibody of claim 17 or 18, which binds to the VRPI with greater affinity than to another isoform of the VRPI.

20. The antibody of claim 17 or 18, which binds to the VRPI with greater affinity than to any other isoform of the VRPI.

21. A kit comprising the antibody of claim 17 or 18.

22. A kit comprising a plurality of antibodies, wherein each antibody comprising the plurality of antibodies is an antibody of claim 17 or 18.

23. A pharmaceutical composition comprising a therapeutically effective amount of an antibody of claims 17 or 18, and a pharmaceutically acceptable carrier.

24. A method of treating or preventing Vascular Response comprising administering to a subject in need of such treatment or prevention a therapeutically effective amount of one or more of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-183, or an ortholog thereof.

25. A method of treating or preventing Vascular Response comprising administering to a subject in need of such treatment or prevention a therapeutically effective amount of an antibody as claimed in any one of claims 17 or 18.

26. A method of treating or preventing Vascular Response comprising administering to a subject in need of such treatment or prevention a therapeutically effective amount of a nucleic acid encoding one of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-183, or an ortholog thereof

27. A method of treating or preventing Vascular Response comprising administering to a subject in need of such treatment or prevention a therapeutically effective amount of a nucleic acid that inhibits the function of one or more of the following Vascular Response-Associated Protein Isoforms (VRPIs): VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-216, VRPI-238, VRPI-245, VRPI-249, VRPI-269, or an ortholog thereof.

28. The method of claim27, wherein the nucleic acid is a VRPI antisense nucleic acid or ribozyme.

29. A method of screening for agents that interact with a VRPI, a VRPI fragment, or a VRPI-related polypeptide, said method comprising:
(a) contacting a VRPI, a VRPI fragment, or a VRPI-related polypeptide with a candidate agent; and
(b) determining whether or not the candidate agent interacts with the VRPI, the VRPI fragment, or the VRPI-related polypeptide.

30. The method of claim 29, wherein the VRPI, the VRPI fragment, or the VRPI-related polypeptide is expressed by cells.

31. The method of claim 30, wherein the cells express a recombinant VRPI, a recombinant VRPI fragment, or a recombinant VRPI-related polypeptide.

32. A method of screening for agents that modulate the expression or activity of a VRPI, a VRPI fragment or a VRPI-related polypeptide comprising:
(a) contacting a first population of cells expressing a VRPI, a VRPI fragment or a VRPI-related polypeptide with a candidate agent;
(b) contacting a second population of cells expressing said VRPI, said VRPI fragment or said VRPI-related polypeptide with a control agent; and
(c) comparing the level of said VRPI, said VRPI fragment or said VRPI-related polypeptide or mRNA encoding said VRPI, said VRPI fragment or said VRPI-related polypeptide in the first and second populations of cells, or comparing the level of induction of a cellular second messenger in the first and second populations of cells.

33. The method of claim 32, wherein the level of said VRPI, said VRPI fragment or said VRPI-related polypeptide, mRNA encoding said VRPI, said VRPI fragment or said VRPI-related polypeptide, or said cellular second messenger is greater in the first population of cells than in the second population of cells.

34. The method of claim 32, further comprising the step of determining the ability of the candidate agent to induce an unwanted vascular response.

35. The method of claim 32, wherein the level of said VRPI, said VRPI fragment or said VRPI-related polypeptide, mRNA encoding said VRPI, said VRPI fragment or said VRPI-related polypeptide, or said cellular second messenger is less in the first population of cells than in the second population of cells.

36. A method of screening for or identifying agents that modulate the expression or activity of a VRPI, a VRPI fragment or a VRPI-related polypeptide comprising:
(a) administering an agent to a first mammal or group of mammals; and
(b) comparing the level of expression of the VRPI, the VRPI fragment or the VRPI-related polypeptide or of mRNA encoding the VRPI, the VRPI fragment or the VRPI-related polypeptide in the first group with that of a second group which has not been administered the agent, or comparing the level of induction of a cellular second messenger in the first and second groups.

37. The method of claim 36, wherein the mammals are animal models for Vascular Response.

38. The method of claim 36 or 37, wherein the level of expression of said VRPI, said VRPI fragment or said VRPI-related polypeptide, mRNA encoding said VRPI, said VRPI fragment or said VRPI-related polypeptide, or of said cellular second messenger is greater in the first group than in the second group.

39. The method of claim 36 or 37, wherein the level of expression of said VRPI, said VRPI fragment or said VRPI-related polypeptide, mRNA encoding said VRPI, said VRPI fragment or said VRPI-related polypeptide, or of said cellular second messenger is less in the first group than in the second group.

40. The method of claim 36 or 37, wherein the levels of said VRPI, said VRPI fragment or said VRPI-related polypeptide, mRNA encoding said VRPI, said VRPI fragment or said VRPI-related polypeptide, or of said cellular second messenger in the first and second groups are further compared to the level of said VRPI, said VRPI fragment or said VRPI-related polypeptide or said mRNA encoding said VRPI, said VRPI fragment or said VRPI-related polypeptide in normal control mammals.

41. The method of claim 36 or 37, wherein administration of said candidate agent modulates the level of said VRPI, said VRPI fragment or said VRPI-related polypeptide, or said mRNA encoding said VRPI, said VRPI fragment or said VRPI-related polypeptide, or said cellular second messenger in the first group towards the levels of said VRPI, said VRPI fragment or said VRPI-related polypeptide or said mRNA or said cellular second messenger in the second group.

42. The method of claim 36 or 37, wherein said mammals are human subjects having Vascular Response.

43. A method of screening for or identifying agents that interact with a VRPI, a VRPI fragment or a VRPI-related polypeptide, comprising
(a) contacting a candidate agent with the VRPI, the VRPI fragment or the VRPI-related polypeptide, and
(b) detecting binding, if any, between the agent and the VRPI, the VRPI fragment or the VRPI-related polypeptide.

44. A method of screening for or identifying agents that modulate the activity of a VRPI, said VRPI fragment or a VRPI-related polypeptide, comprising
(a) in a first aliquot, contacting a candidate agent with the VRPI, said VRPI fragment or the VRPI-related polypeptide, and
(b) comparing the activity of the VRPI, said VRPI fragment or the VRPI-related polypeptide in the first aliquot after addition of the candidate agent with the activity of the VRPI, said VRPI fragment or the VRPI-related polypeptide in a control aliquot, or with a previously determined reference range.

45. The method according to claim 43 or 44, wherein the VRPI, the VRPI fragment or the VRPI-related polypeptide is recombinant protein.

46. The method according to claim 43 or 44, wherein the VRPI, the VRPI fragment or the VRPI-related polypeptide is immobilized on a solid phase.

47. A method for screening, diagnosis or prognosis of Vascular Response in a subject or for monitoring the effect of an anti-Vascular Response drug or therapy administered to a subject, comprising:
(a) contacting at least one oligonucleotide probe comprising 10 or more consecutive nucleotides complementary to a nucleotide sequence encoding a VRPI chosen from, VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269, or an ortholog thereof, with an RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

48. The method of claim 47, wherein step (a) comprises contacting a plurality of oligonucleotide probes comprising 10 or more consecutive nucleotides complementary to a nucleotide sequence encoding a VRPI chosen from, VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269, or an ortholog thereof, with an RNA obtained from a biological sample from the subject or with cDNA copied from the RNA wherein said contacting occurs under conditions that permit hybridization of the probe to the nucleotide sequence if present.

49. The method of claim 48, wherein step (a) includes the step of hybridising the nucleotide sequence to a DNA array, wherein one or more members of the array are the probes complementary to a plurality of nucleotide sequences encoding distinct VRPIs.

50. A method of modulating the activity of one or more of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269, or an ortholog thereof, comprising the step of contacting a cell with an agent which specifically binds to one or more of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269, or an ortholog thereof, whereby the activity of the respective VRPI is modulated.

51. An agent that modulates the activity of one or more of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269, or an ortholog thereof, wherein said agent is identified by the method of any of claims 36, 43 or 44.

52. An agent as claimed in claim 51 for use in the manufacture of a medicament for the treatment or prevention of Vascular Response.

53. A pharmaceutical composition, comprising:the agent of claim 51 or 52, and a pharmaceutically acceptable carrier.

54. A method of treating or preventing Vascular Response comprising administering to a subject in need of such treatment or prevention a therapeutically effective dose of an agent that modulates the activity of one or more of the following Vascular Response-Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269, or an ortholog thereof, whereby the symptoms of Vascular Response are ameliorated.

55. A method for identifying targets for therapeutic modulation of Vascular Response wherein the activity of one or more of the following Vascular Response -Associated Protein Isoforms (VRPIs): VRPI-1, VRPI-14, VRPI-18, VRPI-19, VRPI-23, VRPI-28.1, VRPI-28.2, VRPI-32.1, VRPI-32.2, VRPI-36, VRPI-38, VRPI-43, VRPI-47.1, VRPI-47.2, VRPI-58, VRPI-61, VRPI-63, VRPI-65, VRPI-68.1, VRPI-68.2, VRPI-72, VRPI-73, VRPI-75, VRPI-86, VRPI-93, VRPI-94, VRPI-95, VRPI-100, VRPI-102, VRPI-104, VRPI-106, VRPI-108, VRPI-115, VRPI-116, VRPI-118.1, VRPI-118.2, VRPI-120, VRPI-130, VRPI-183, VRPI-216, VRPI-238, VRPI-245, VRPI-249, VRPI-269, or an ortholog thereof, is utilized as a measure to determine whether a candidate target is effective for modulation of Vascular Response.
